SEQUENCE LISTING (11 sequences)

SEQ ID NO: 1 - protein, 4 aa, Artificial Sequence
  Description of Artificial Sequence Synthetic peptide
NYTA

SEQ ID NO: 2 - protein, 151 aa, Artificial Sequence
  Description of Artificial Sequence Synthetic polypeptide
EVKALCVKSLEGVKVGTGDEAKQNGIDFYKYMFTNYPDLRVYFKGAENYTAEDVQKSERFAKQGQRILLACHILANTYDDEETFNAYVRETVNRHRIFKMDPELWTAFWTVFVGFLESKGTLDEETKAAWMQIGKEFAEEAQSHLKSLGLP

SEQ ID NO: 3 - protein, 149 aa, Ascaris suum
NKTRELCMKSLEHAKVDTSNEARQDGIDLYKHMFENYPPLRKYFKNREEYTAEDVQNDPFFAKQGQKILLACHVLCATYDDRETFNAYTRELLDRHARDHVHMPPEVWTDFWKLFEEYLGKKTTLDEPTKQAWHEIGREFAKEINKHGR

SEQ ID NO: 4 - protein, 170 aa, Ascaris suum
AVRHQCMRSLQHIDIGHSETAKQNGIDLYKHMFENYPSMREAFKDRENYTAEDVQKDPFFVKQGQRILLACHLLCASYDDEETFHMYVHELMERHERLGVQLPDQHWTDFWKLFEEFLEKKSHLCEHTKHAWAVIGKEFAYEATRHGKEHHEHKEEHKEEHKEEHKEEQH

SEQ ID NO: 5 - protein, 162 aa, Trichuria canis
SFGSCTVSAESPRELCMKSLKDVHVGTGDHAKQCGIDLYKHMFEHYPDLRKFFKNRENYTAEDVQKDEFFAKQGQKILLACHVLCATVDDMETFNAYSHEVLDRHERDHVELPASAWTDFWKIFIEYLEKKMTVDEATKNAWMEIGKDFATAVGQHESSHSH

SEQ ID NO: 6 - protein, 159 aa, Onchocerca volvulus
FEMSHSGTKAKCLKVMNESGRVGNDDAAKQDGLNFYKYMFGHHPDLRVYFKGAENFTPTDVQNSDRFAKQGQRVLLAVRILINTYDDSETFKAYARETVNRHIKFKMDRTLWAKFFTLFVNNLKEHTTVDEETEKAFQQIGKEFSDECIKHTVALNLPN

SEQ ID NO: 7 - protein, 153 aa, Strongyloides stercoralis
KMDSTEVKKVARKSLEVLRLDDEHGAIPHGKDFYKFMFGNYPQLRVYFKGAENYTPEQVGASERFAKQGVRIMLAMHILATAYDDQPTFLAYVRETINRHRIFKMPEELWDAFFPVWLGFLQTKGVVTPEVTNAWNQIGKTFTSEAHRYMKSN

SEQ ID NO: 8 - protein, 144 aa, Anchylostoma caninum
LETVAIFPGPKEVQNAKDFYKYMFTHHPDLRRYFKGAENFTAEDVQKSERFDKQGQRILLAVYILADTFDDEPTFRAYARETVNRHRQFKMDPELWSAFFTVYVNFLASRGPLSDEQKKAWAQLGKVFDEECQSHLKELGLPHC

SEQ ID NO: 9 - protein, 152 aa, Nector americanus
KKHTVASLGVVPVGKTPDKIQNGIDFYKYFFTNHPEARKYFKGAENFTADDVQKSERFEKQGNALLLSVHILANTYDNEEVFRAYCRDHTNRHAAKGIDPHYWKSFWGIWMGFLESKGATVSGDQKAAWETLGTMFNEECQSHLSKLGLPHA

SEQ ID NO: 10 - protein, 166 aa, Heterodera glycines
KNNCSAKSPKMSTREEIQAVCVQSLKQLKIGVGEEELQNGKDFYKFFFTNYPTLRVYFKGAEKYEAEDVQKSERFEKQGQRILVAMHLIANVYSNEMLFNAYVRETVNRHRQFKMEPSLWKAFWTVWTGFMETKVDLTEQAKSAWMSLGEDFAKEALAHLKRLGIP

SEQ ID NO: 11 - protein, 159 aa, Caenorhabditis elegans
MSMNRQEISDLCVKSLEGRMVGTEAQNIENGNAFYRYFFTNFPDLRVYFKGAEKYTADDVKKSERFDKQGQRILLACHLLANVYTNEEVFKGYVRETINRHRIYKMDPALWMAFFTVFTGYLESVGCLNDQQKAAWMALGKEFNAESQTHLKNSNLPHV

(19) United States
(12) Patent Application Publication
(10) Pub. No.: US 2011/0008381 A1
(43) Pub. Date: Jan. 13, 2011

(54) SUPPRESSION OF ALLERGIC INFLAMMATION BY ASCARIS HEME-BINDING PROTEIN (HBP)

(76) Inventors: Andrea Keane-Myers, Rockville, MD (US); Virgilio G. Bundoc, Washington, DC (US); Marcus G. Hodges, Bethesda, MD (US); Shweta Trivedi, Durham, NC (US); Satish K. Madala, Bethesda, MD (US); Thomas A. Wynn, Bethesda, MD (US); Joseph F. Urban, Beltsville, MD (US)

Correspondence Address: OTT- NIH;c/o WOODCOCK WASHBURN LLP, CIRA CENTRE, 12TH FLOOR, 2929 ARCH STREET, PHILADELPHIA, PA, 19104-2891, US

(21) Appl. No.: 12/600,363

(22) PCT Filed: May 19, 2008
(86) PCT No.: PCT/US2008/006367

§ 371 Date: Jun. 23, 2010

Related U.S. Application Data
(60) Provisional application No. 60/924,537, filed on May 18, 2007.

(51) Int. Cl. A61K 39/00; A61P 37/06; A61P 37/08

(52) U.S. Cl. 424/191.1

(57) ABSTRACT

This invention relates, e.g., to a method for suppressing inflammation (e.g. allergic inflammation, for example asthma caused by ragweed), a Th1-mediated condition and/or a Th2-mediated condition and/or a condition in which administration of an antioxidant would be beneficial, in a subject in need thereof, comprising administering to the subject an anitinflammatory-effective amount of Ascaris-derived heme-binding protein (HBP) or an active fragment or variant thereof (e.g. domain 2 of the polypeptide or an active variant thereof). Also described are pharmaceutical compositions for suppressing one of the above conditions, adjuvant compositions and immunogenic compositions comprising Ascaris-derived heme-binding protein (HBP), or an active fragment or variant thereof, and an antigen.

This application claims the benefit of the filing date of U.S. provisional application 60/924,537, filed May 18, 2007, which is incorporated by reference herein in its entirely.

### FIELD OF THE INVENTION

This invention relates, e.g., to methods for treating inflammation, such as allergic inflammation.

### BACKGROUND INFORMATION

Allergic asthma is characterized by antigen-specific IgE production, reversible airway hyper-reactivity and eosinophilic infiltration of the airways. A number of studies have demonstrated a dramatic increase in the prevalence of allergic disorders in emerging and industrialized countries. These studies suggest that the hygienic Westernized environment may lack an allergy-protective mechanism or infection.

Recent studies have found that infection by helminthic parasites may negatively impact the development of allergic disease. Helminth infection currently affects over 2 billion people world-wide, causing significant morbidity. The most successful geohelminths are members of the Ascaris species (e.g., Ascaris lumbricoides (A. lumbricoides) and Ascaris suum (A. suum)), which are known to infect 1.5 billion people. Helminthic parasites modulate host immune responses to ensure chronic infection and survival in their host by down regulating inflammatory responses. Some of the present inventors and their colleagues have studied chronic A. suum infection in a murine model of ragweed-induced allergic conjunctivitis and allergic asthma, and have demonstrated that chronic A. suum infection prevents allergic inflammation in sites distal from larval migration. (Schopf et al. (2005) Invest Ophthalmol Vis Sci. 46, 2772-80.). This protection was due, in part, to the induction of immunoregulatory cytokines such as IL-10. These observations suggested that products derived from Ascaris Helminthes, either alone or in combination, may offer a means for treating allergies, autoimmune diseases, or the like.

In recent studies, the inventors and colleagues used exposure to a cocktail of A. suum antigens from the pseudoceolomic fluid (PCF) of A. suum in lieu of Ascaris infection (McConchie et al. (2006) Infect Immun. 74, 6632-41). Administration of PCF during ragweed (RW) sensitization significantly reduced eosinophil migration into the conjunctiva, pulmonary eosinophilic inflammation, and total lung pathology in response to RW challenge. Furthermore, PCF exposure reduced the secretion of the pro-allergic cytokines IL-5 and IL-13 in the broncho-alveolar lavage fluid (BALF) after RW exposure. These findings suggested that Ascaris-derived PCF is capable of suppressing the allergic response to a traditional allergen and at multiple tissue sites.

Toll-like receptors (TLRs) on dendritic cells and other antigen presenting cells recognize specific molecular patterns on invading pathogens, leading to the development of host immunity. A number of pathogens, including helminths, viruses, and bacteria, have used pattern recognition by TLRs to modulate host immunity and inflammation to establish a chronic infection. The molecular basis for the TLR-induced tolerance induction observed in individuals infected with helminth parasites is not well understood.

### DESCRIPTION OF THE DRAWINGS

FIG. 1 shows the purification and characterization of native Ascaris suum hemoglobin (AsHb). In FIG. 1A, pseudocoelomic fluid was separated into two major fractions on a Superose 6 gel filtration column. The peak corresponding to AsHb was dialyzed against Q-sepharose loading buffer (25 mM Tris-HCl pH 8, 25 mM NaCl and 1 mM EDTA) before loading onto Q HP 5 ml anion exchange column. In FIG. 1B, bound proteins were eluted using a salt gradient (indicated by the dashed line) and peak corresponding AsHb is indicated by an arrow. The protein identity was confirmed using mass spectrometry analysis of the purified protein. In FIG. 1C, purified native AsHb was separated using 2D gel electrophoresis; the about 42 kDa protein band corresponds to AsHb polypeptide. In FIG. 1D, purified native and recombinant AsHb was separated on 12% SDS-PAGE and transferred to a nitrocellulose membrane. Lane 1: Molecular weight markers; Lane 2: Purified native AsHb; Lane 3: Purified recombinant AsHb. The color was developed using a DAB substrate kit (BD Pharmingen).

FIG. 2 shows that AsHb interacts and modulates cytokine production in bone marrow derived dendritic cells (BMDC). In FIG. 2A, BMDC from WT C57BL/10 mice were stimulated with pseudocoelomic fluid (PCF; 100 mg/ml), purified Ascaris hemoglobin (AsHb; 10 μg/ml) and the low molecular weight fraction (LMW; 10 μg/ml) of PCF alone or were costimulated with LPS (50 ng/ml) for 12 h. Culture supernatants were analyzed for cytokines using a multipex cytokine analysis method. ***p<0.001; **p<0.01; *p<0.05 compared to LPS alone treated cells. #p<0.01compared to cells treated with media alone. FIG. 2B shows that BMDC recognizes and internalize AsHb. WT C57BL/10 BMDC were pulsed with or without AsHb and AlexaFluor 594-conjugated dextran (upper panel), -transferrin (lower panel), or AsHb alone (lower panel) for 30 minutes. Cells were then fixed and stained with anti-AsHb (upper and lower panels) as described in the materials and methods. The co-localization of AsHb with transferrin is indicated by white arrows in the merge panel. Differential interference contrast (DIC) images are shown in grey. scale bar, 5 μm. Small arrows show the co-localization of AsHb with transferrin The above panels were one representative of four independent experiments.

FIG. 3 shows the characterization of AsHb. In FIG. 3A, the secondary structure of AsHb was analyzed using Gamier and Robson to indicate alpha and beta regions in the primary sequence as shown on the top of panel corresponding to the protein sequence numbers. The Kyte and Dolittle hydrophilicity plot indicates AsHb is rich in hydrophilic regions and has three discrete hydrophobic regions. The domain composition shown at the bottom of the panel indicates the presence of a hydrophobic signal sequence at the N-terminal side followed by two heme binding domains arranged in tandem. In FIG. 3B, a clustal W multiple sequence alignment of AsHb was generated between domain1 (SEQ ID NO:3) and domain 2 (62% homology) (SEQ ID NO:4) and their nearest homolog sequences from seven other nematode species Toxocara canis (68%) (SEQ ID NO:5), Onchocerca volvulus (45%) (SEQ ID NO:6), Strongyloides stercoralis (40%) (SEQ ID NO:7), Ancylostoma caninum (40%) (SEQ ID NO:8), Nector americanus (39%) (SEQ ID NO:9), Heterodera glycins (39%) (SEQ ID NO:10) and Caenorhabditis elegans (41%) (SEQ ID NO:11). The residues that match to A. suum domain1 are shaded to indicate the identity. The consensus sequence in the alignment is indicated on the top of the panel (SEQ ID NO:2). The residue numbers for each sequence is indicated on the right side of the panel. Potential N-glycosylation sites were predicted using NetNGlyc 1.0 and are highlighted with a box. Conserved amino acids are numbered as positions in the domains of human hemoglobin. Charged C-terminal tail in Ascaris Domain 2 is indicated with a dotted line. In FIG. 3C, a bayesian phylogenetic tree for all heme binding domains in nematodes were created using the MRBAYNES program. In addition to the sequences used in the alignment in FIG. 3B, the following species sequences were also included, Caenorhabditis briggsae, Caenorhabditis cremanei and Pseudoterranova decipiens. The number next to the nodes indicates the probability value for those branches. Branch lengths represent mean values of the sampled trees with a scale bar 0.2 amino acid substitutions per site.

FIG. 4 shows that the domain 2 (rD2) of AsHb is critical in modulating the cytokine production in dendritic cells. FIG. 4A shows the purification of recombinant AsHbs. SDS-PAGE for the purified recombinant AsHb fractions. Lane 1, Mol Wt Standards; Lane2, Total soluble extract for domain1; Lane3, 4 & 5, Purified recombinant Domain1 (rD1), Domain (rD2) and full length AsHb without a signal peptide (r AsHb), respectively. In FIG. 4B, CD11c+ve BMDC from C57BL/10 mice were costimulated with 0.5 μM purified recombinant AsHb (r AsHb), Domain1 (rD1) or Domain 2 (rD2) and LPS (50 ng/ml) for 12 h. Culture supernatants were analyzed for the cytokines IL-10, IL-12 and TNF-α. ** p<0.01; *p<0.05 compared with LPS alone treatment. This is a representative of three independent experiments.

FIG. 5 shows that AsHb is a secretory and antigenic protein of A. suum. FIG. 5A shows an immunoblot using a 1:5000 dilution of rabbit polyclonal anti-domain1 (AsHb) antibodies. Lane 1, Purified native AsHb; Lane 2, Excretory/secretory product from adult A. suum; Lane 3, Excretory/secretory product from L3/L4 larvae; Lane 4, Muscle extract from adult A. suum; Lane 5, Intestinal extract from adult A. suum. FIG. 5B shows AsHb specific immunohistochemical staining of cross sections of eggs and larval/adult parasites harvested from the lungs of A. suum infected pigs at day 7, and the small intestines at day 10 and day 14 of post infection.

FIG. 6 shows the measurement of AsHb specific class and subclass antibody responses. Diluted serum antibodies from A. suum infected or uninfected mice were added to AsHb-coated (0.5 μg/well) micro plate wells and were incubated with avidin-conjugated anti-IgG1, anti-IgG2a and anti-IgE. Absorbance at 450 nm corresponds to the levels of antibody present in a given sample. Optical density values were expressed in arbitrary units. This is a representative of three individual experiments.

FIG. 7 shows that AsHb attenuates superoxide and reactive oxygen species generation. BAL cells were prepared from A. summ infected or uninfected pigs and stimulated with opsonized bacteria in the absence or presence of AsHb or superoxide dismutase (SOD) as indicated above. Formation of superoxides and other reactive oxygen anions is quantified as a measure of cytochrome C reductants generated using a calorimetry based assay.

FIG. 8 shows that AsHb activates dendritic cells. CD11c +ve BMDCs from WT C57BL/10 mice were treated with purified native AsHb (10 μg/ml) alone or costimulated with LPS (50 ng/ml) for 12 h. The shaded area represents unstimulated cells. Cells stimulated with AsHb, LPS or both are represented with a dark line. Mean fluorescence intensity values for CD40, CD86 and MHC II are indicated in the histogram plots.

FIG. 9 shows the nature of the AsHBb glycan, which was analyzed by subjecting AsHb to deglycosylation with various endoglycosidases. The purified native AsHb was treated with N-Glycosidase F (PNGaseF), endoglycosidase H (EndoH) and endoglycosidase D (EndoD) according to the manufacturer's recommendations. Prior to the treatment with the enzymes, AsHb (0.5 mg/ml) was denatured in the denaturation buffer (0.5% SDS and 40 mM DTT) for 5 minutes at 100° C. and cooled. Thus, denatured AsHb (40 μl) was mixed with 5 μl of 10× reaction buffer for PNGaseF (0.5 M sodium phosphate buffer pH7.5), EndoH (0.5 M sodium citrate, pH 5.5) and EndoD (0.2M Tris-HCL pH 7.5 and 0.2% BSA). Samples are then digested with 5 μl of enzymes for 4 h before being analyzed by 12% SDS-PAGE. Lane 1, Biorad broad range molecular weight marker; Lane 2, Purified native AsHb alone; Lane 3, 4, and 5 AsHb treated with endoglycosidases PNGaseF, endoglycosidaseD and endoglycosidaseH, respectively. Glycosylated or deglycosylated AsHb is indicated by an arrow.

FIG. 10 shows schematically the time course of an experiment to show that HBP can act as an adjuvant for anthrax vaccines.

### DESCRIPTION OF THE INVENTION

To identify and characterize the components of pseudocelomic fluid (PCF) that play a role in modulating allergic responses to allergen challenge, as well modulating Th-1-associated conditions, we screened PCF from A. suum for antigenic and immunomodulatory proteins. We describe herein the identification of a heme binding protein (HBP) from A. suum. This protein is sometimes referred to herein as “HBP,” but a skilled worker will recognize that an “Ascaris-derived HPB” is meant. HBP is sometimes referred to herein as Ascaris suum hemoglobin, or “AsHb.” The HBP is shown to be an about 42 KDa glycoprotein comprising two globin-like domains; to be an antigenic protein that can modulate activation and maturation of bone marrow derived dendritic cells (BMDC) in response to stimuli with bacterial lipopolysaccharide (LPS), to stimulate dendritic (DC) to produce significant increases in IL-10 but not IL-12 upon co-stimulation with LPS, and to reduce free radical formation by host protective cells. The differences between IL-10 and IL-12 cytokine production following costimulation with HBP and LPS were abrogated in genetically deficient TLR4 and MyD88, but not TLR2, mice, suggesting that HBP augments TLR4 dependent IL-10 production. Serum from pigs chronically infected with A. suum demonstrated immune recognition to purified HBP. Polyclonal antibodies raised against HBP demonstrated the expression of HBP in the muscle and intestinal tissues and the excretory/secretory products obtained from third and fourth-stage larvae (L3/L4) and adult worms. Analysis of secretory/excretory products from adult and L3/L4 larvae of A. suum indicates that HBP is a major secretory protein. We expressed and purified HBP, as well as its two domains, domain 1 and domain 2, and showed that HBP ability to activate BMDC is dependent on domain 2 but not domain 1.

Unexpectedly, we found that, whereas unfractionated PCF suppresses DC activation and cytokine production and prevents stimulation of DC by agents such as LPS, purified HBP exhibits very different properties: it activates DC (but, in the presence of agents like LPS, does not cause additional activation) and it enhances cytokine production and, in particular, enhances IL-10. PCF administration prevents an initial response from occurring, as it inhibits the initiation of the inflammatory cascade. By contrast, HBP can activate DC and alter cells such that they ultimately suppress responses through the production of IL-10 and can therefore act on the effector phase of the inflammatory response (i.e. modulate a response that is already occurring). The findings reported here demonstrate a novel mechanism of TLR4-mediated response to Ascaris suum-derived HBP, which results in altered cytokine responses in antigen presenting cells (APC) such as DC. Because activation and cytokine production by APC is the first step in the development of the allergic response, alteration of APC function would be expected to prevent the development of allergic disease and other autoimmune diseases. Thus, the findings reported herein suggest that Ascaris-derived HBP, in particular domain 2 of the protein, can be used to suppress inflammation (e.g., allergic inflammation or Th-1-mediated inflammation) in a subject. In addition, the demonstration herein that HBP can remove extracellular free radical reductants produced by Ascaris-activated swine bronchial alveolar cells further supports the use of HBP to suppress inflammation, and particularly indicates that HBP can be used for treating inflammatory conditions such as colitis.

Unexpectedly, in contrast to PCF, HBS activates DCs, as evidenced by upregulation of activation cell markers, and thus would be expected to function as an adjuvant, whereas PCF would not.

Advantages of using Ascaris-derived HBP or an active fragment or variant thereof to suppress inflammation (e.g., inflammatory diseases or conditions, including allergic inflammation, Th-2-associated or Th-1-associated conditions) include that the protein molecules are relatively inexpensive and easy to produce. Importantly, the invention provides a way to treat allergic diseases or prevent allergic reactions, rather than merely ameliorating the symptoms.

Furthermore, the findings reported here (particularly the demonstration of TLR4-dependent IL-10 production in DCs costimulated with LPS) suggest that HBP or an active fragment or variant thereof, can serve as a TLR-4 adjuvant. This suggests that HBP can be used to augment vaccines directed against infection by bacteria or other infectious agents, for which the immunogenic response is mediated by TLR4. These findings also suggest that HBP may be valuable as a stand-alone immunomodulator.

Although it may seem paradoxical that the same polypeptide can suppress immunological reactions (e.g. suppress allergic inflammation) under some circumstances, and enhance immunological reactions (e.g., act as an adjuvant) under different circumstances, this paradox is readily resolvable. Without wishing to be bound by any particular mechanism, it is suggested that HBP causes very fast activation of dendritic cells (as can be seen by increased activation marker expression upon exposure), so it can act as a potent adjuvant when co-administered with an entity that initiates an inflammatory response. However, HBP can also induce DC to produce IL-10 hours after the activation and then act via a negative feedback mechanism to stop an ongoing effector response, since an ongoing reactionary inflammatory response may have ultimately more deleterious physiologic consequences for the host than the initiation of the disease, itself. Thus, HPB can prevent an immune response in an animal during an ongoing inflammatory response. In essence, whether the adjuvant effect or the suppressive effect wins out depends on the microenvironment of the cells.

One aspect of the invention is a method for suppressing inflammation (e.g., an inflammatory condition or disease) in a subject in need thereof, comprising administering to the subject an anti-inflammatory-effective amount of the polypeptide, Ascaris-derived heme-binding protein (HBP), or an active fragment or variant thereof. The suppression may be mediated, e.g., by the anti-oxidant activity of the polypeptide and/or by its stimulation of IL-10 and modulation of other cytokines.

A method of the invention can be a method for suppressing a Th-2-associated disease or condition (a Th-2 driven pathology), including any of a variety of types of allergic inflammation, for example allergic asthma (e.g., caused by ragweed, including severe allergic asthma), allergic conjunctivitis, allergic dermatitis, allergic eczema, allergic rhinitis, a food allergy, an eosinophil-associated gastrointestinal disorder, hyper eosinophilic syndrome (HES), eczema, or chronic urticaria.

A method of the invention can be a method for suppressing a Th-1-associated disease or condition, which include, but are not limited to, autoimmune disorders, such as inflammatory bowel disease (IBD) or multiple sclerosis (MS).

A method of the invention can be a method for suppressing Th-1- or Th-2-associated intestinal inflammatory disorders, such as Crohn's disease (CD) and ulcerative colitis (UC), popularly known as inflammatory bowel disease (IBD). The anti-oxidant activity of HBP can also be use for treating conditions associated with ageing, e.g., it can be used in an anti-wrinkle cream or to treat stress in the brain (e.g., forms of dementia).

A method of the invention can be a method for suppressing a marker of an immunosensitive property of a cell or tissue in vitro.

The polypeptide which is administered in a method of the invention can, e.g., comprise domain 2 of Ascaris-derived HBP; and/or it can be produced by a synthetic or recombinant molecular procedure.

Another aspect of the invention is a pharmaceutical composition comprising an amount of an HBP polypeptide, or an active fragment or variant thereof, that is effective to suppress inflammation (e.g., allergic inflammation) and a pharmaceutical carrier.

Another aspect of the invention is a kit for treating inflammation (e.g. allergic inflammation, a Th-1-associated disease, a Th-2-associated disease, etc.) comprising an anti-inflammatory effective amount of an HBP polypeptide or an active fragment or variant thereof, or of a pharmaceutical composition of the invention, optionally in a vessel.

Another aspect of the invention is an adjuvant composition comprising an amount of Ascaris-derived HBP or an active fragment of variant thereof that is effective to elicit an adjuvant effect (a measurable amount of an adjuvant effect). The HBP or active fragment or variant thereof may be isolated or purified from an Ascaris organism, or it may be produced by a synthetic or recombinant molecular procedure.

Another aspect of the invention is an immunogenic composition comprising an adjuvant composition of the invention and an antigen. The antigen may be selected from, e.g., peptides, proteins, toxoids, glycoproteins, glycolipids, lipids, carbohydrates and/or polysaccharides; it may be derived from a biologic or infectious organism of the animal or plant kingdom; it may be whole or disrupted microorganisms, including viruses, bacteria or parasites, attenuated and/or inactivated; and/or it may be produced by synthetic or recombinant molecular procedures.

Another aspect of the invention is a method for inducing an immune response, comprising administering to a subject in need thereof an effective amount of an immunogenic composition as above.

The Examples herein are directed primarily to HBP-derived from (isolated from) A. suum (a porcine species). However, the genome of the related human Ascaris species, A. lumbricoides, shares greater than 95% homology with the genome of A. suum and shares many of its properties (e.g., the two species are cross-infectious of host species). The sequences of the HBPs from the two species would be expected to share nearly complete identity, and they would be expected to exhibit nearly identical properties. Therefore, we refer to the HBP described herein as being “Ascaris-derived,” indicating that it can be derived (isolated) from either A. suum or A. lumbricoides. Furthermore, a variety of other parasitic helminthes exhibit properties similar to Ascaris. For example, the whole organisms, when present in their hosts, appear to suppress allergic inflammation. Thus, the HBPs from these parasites would also be expected to be similar to Ascaris-derived HBP. These other parasitic nematodes include, e.g., Brugia malayi, Heligmosomoides polygyrus, Nipostrongylus brasiliensis, Necator americanus, Trichuris muris, Trichuris trichiura, Toxocara species, including dog, cat and sea mammal Ascarids, including Anisakis and Phochynema, Onchocerca volvulus, Strongyloides stercoralis, Ancylostoma caninum, Ancylostoma duodenale and Wuchereria bancrofti.

As used herein, the singular forms “a,” “an,” and “the” include plural referents unless the context clearly dictates otherwise. For example, “an” active fragment, as used above, means one or more active fragments, which can be the same or different.

A “subject,” as used herein, can refer to any animal which has or can have an inflammatory disease or condition (e.g., allergic inflammation, a Th-1-associated condition, or a form of colitis), e.g., a mammal, such as an experimental animal, a farm animal, pet, or the like. In some embodiments, the animal is a primate, preferably a human. A subject can also be an animal as above for which it would be beneficial to receive an adjuvated antigen (e.g., in a vaccine).

An “effective amount” of an HBP polypeptide of the invention is an amount that is effective to elicit a measurable amount of biological activity of HBP. For example, an “anti-inflammatory-effective” amount is an amount that can elicit a measurable amount of suppression of an inflammatory reaction, condition or disease. An effective amount of an adjuvant is an amount that can elicit a measurable amount of adjuvant activity.

The terms peptide, polypeptide and protein are used interchangeably herein.

An “active” fragment or variant of an HBP polypeptide of the invention is one which retains a measurable amount of at least one activity of the HBP polypeptide [e.g., the ability to suppress an inflammatory (e.g. allergic) reaction, the ability to act as an antioxidant, or the ability to act as generalized adjuvant]. For example, polypeptides comprising small substitutions, additions, deletions, etc, are tolerated provided they retain such an activity, as are suitable fragments of HBP. Polypeptides that exhibit at least about 90% (e.g., at least about 95%, or at least about 98%) sequence identity to HBP, or to an active fragment thereof, over the entire length of the HBP or active fragment, are also included. Methods for determining if a polypeptide exhibits a particular percent identity to a polypeptide are conventional. Methods for determining if a modified HBP molecules exhibits an activity of HBP are conventional; for example, methods discussed herein can be employed. Of course, tiny fragments such as single amino acids, dipeptides or the like which do not exhibit a measureable amount of an HBP activity are excluded.

Suitable fragments and variants can be designed on the basis, at least in part, of structural and functional studies that have been performed on HBP. See, e.g., Das et al. (2000) Biochemistry 39, 837-42 and Minning et al. (1999) Nature 401, 497-502, and studies described herein. Such studies can provide guidance to the skilled worker as to which sequences are dispensable, and which sequences cannot be altered, when generating active fragments or variants of HBP.

For example, if a full-length HBP molecule is used in a method or composition of the invention, the secretory signals at the N-terminus of the protein can be removed. These sequences are not required for therapeutic HBP activity, since when the polypeptide is administered directly to a subject, there is no need for it to be secreted from the Ascaris organism.

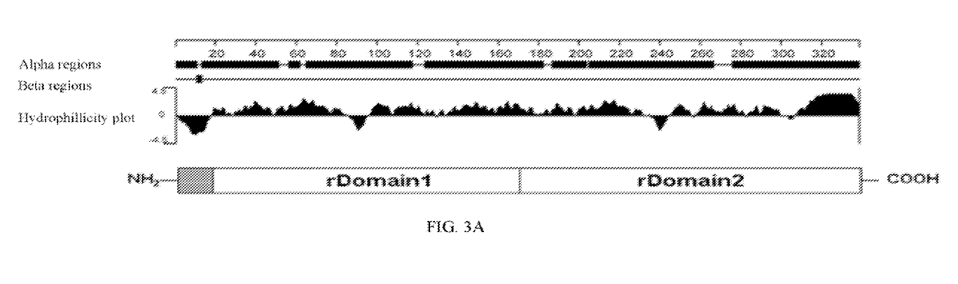

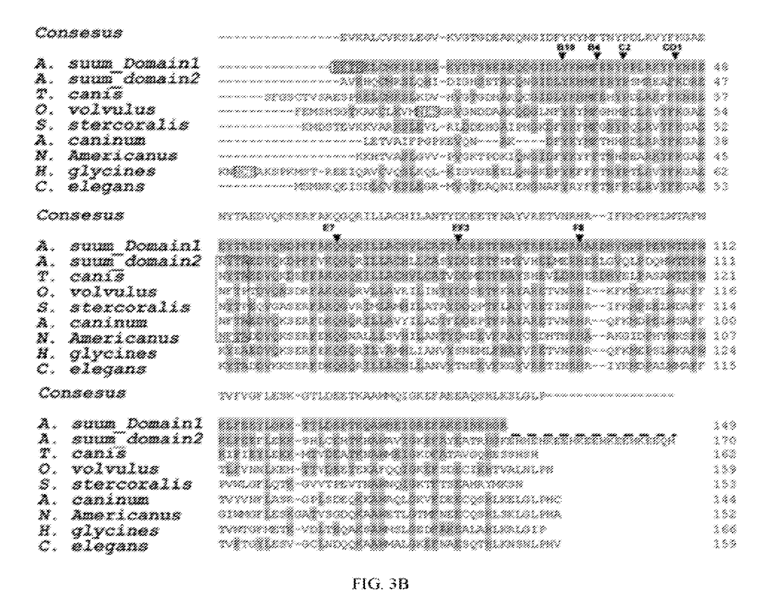

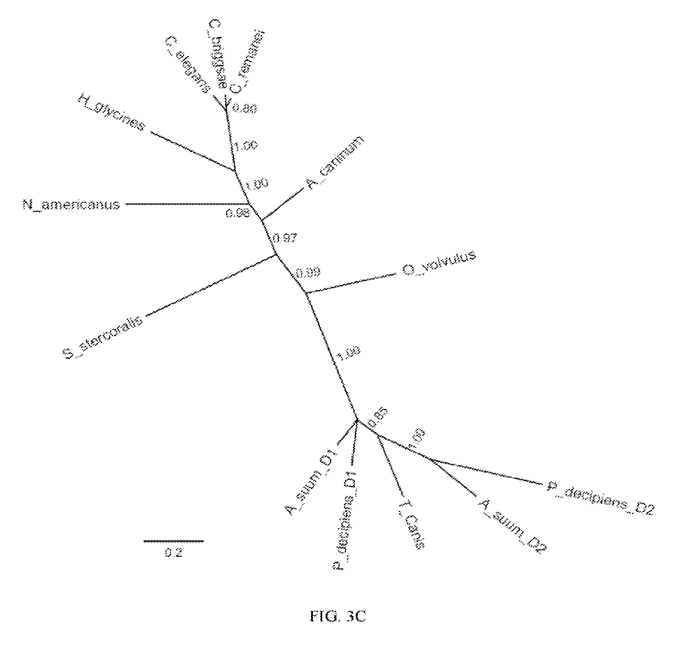

Furthermore, as is shown herein, the immunomodulatory functions of HBP, e.g. the ability of HBP to induce IL-10 in response to LPS stimulation, can be achieved with a fragment of the protein having domain 2 (the C-terminal portion of HBP as shown in FIG. 3A) in the absence of domain 1. Domain 1, by itself, is inactive. Therefore, a fragment of HPB consisting of, or comprising, the sequences of domain 2 can be used in methods and compositions of the invention. Moreover, the molecular characterization of domain 2 suggests that portions of this fragment are important for its activity, and thus can probably not be modified or deleted without reducing its activity, and portions of the molecule are inessential and thus can probably be altered or deleted. For example:

1. The hydrophobic C-terminal tail appears to play a key role in the oligomerization of the fragment (or the intact HBP protein) to a multimer (e.g., a dimer, tetramer or, in the case of natural, full-length HBP, an octamer). This oligomerization appears to be important for the activity of the protein. This conclusion is based on the observation that HBPs from organisms that lack the immunomodulatory activity of Ascaris-derived HBP, such as HBPs from higher organisms and the non-parasitic nematode, C. elegans, fail to oligomerize.

2. Other regions that are unique to domain 2 may contribute to its oligomerization, and thus can likely not be modified or deleted without reducing its activity. See, e.g., Kloek et al. (1993) J Biol Chem 268, 17669-17671 and De Baere et al. (1992) Proc Natl Acad Sci USA 89, 4638-4642.

3. Sequence alignment of domain 1 and domain 2, as shown in FIG. 3B, indicates that that the two domains share 62% similarity. The fact that these two sequences are quite similar, but domain 1 is inactive whereas domain 2 is fully active, allows one to predict which residues are likely to be important for the structure and function of the polypeptide (e.g., for binding the prosthetic group, heme). That is, residues that are unique to domain 2, as indicated by shading in FIG. 3B, may be particularly important for its structure and function, and probably cannot be deleted or altered without affecting function of the protein.

4. The N-glycosylation site in domain 2, having a consensus sequence NYTA (SEQ ID NO:1), appears to be important for its immunomodulatory activity, at least because HBPs from higher eukaryotes and C. elegans, which lack this activity, also lack the N-glycosylation. Without wishing to be bound by any particular mechanism, it is also suggested that the glycosylation may be important for structural stability of the protein and protection from proteases, thus increasing the half-life of the protein. Therefore, this consensus sequence can probably not be deleted or altered without affecting function of the protein.

HBP or an active fragment or variant thereof can be derived (isolated) from Ascaris by conventional methods. For guidance in this and other biochemical, molecular biology, and immunological techniques used for compositions or methods of the invention, see, e.g., Sambrook et al, Molecular Cloning, A Laboratory Manual, current edition, Cold Spring Harbor Laboratory, New York; Miller et al, Genetic Engineering, 8:277-298 (Plenum Press, current edition); Wu et al, Methods in Gene Biotechnology (CRC Press, New York, N.Y., current edition); Methods in Molecular Biology, Vol. 62, (Tuan, ed., Humana Press, Totowa, N.J., current edition); Current Protocols in Molecular Biology, (Ausabel et al, Eds.,), John Wiley & Sons, NY (current edition); Current Protocols in Immunology (Coligan et al., editors, John Wiley & Sons, Inc) and references cited therein.

An “isolated” polypeptide of the invention is in a form other than it occurs in nature, e.g. in a buffer, in a dry form awaiting reconstitution, as part of a kit, etc. The term “isolated,” as used herein, means that the polypeptide is removed from its original environment (e.g., the natural environment if it is naturally occurring), and isolated or separated from most other components with which it is naturally associated. For example, a naturally-occurring polypeptide present in a living host is not isolated, but the same polypeptide, separated from some or all of the coexisting materials in the host, is isolated. Such polypeptides can be part of a composition or reaction mixture, and still be isolated in that such composition or reaction mixture is not part of its natural environment. The term, an “isolated polypeptide,” as used herein, can include 1, 2, 3, 4 or more copies of the polypeptide, and the polypeptide can be in the form of a multimer, such as a dimer, tetramer, octamer, or the like, depending on the particular polypeptide under consideration.

In some embodiments of the invention, the polypeptide is purified. Methods for purifying polypeptides are conventional. See, e.g., the Examples herein. In some embodiments, the polypeptide is substantially purified or is purified to homogeneity. By “substantially purified” is meant that the polypeptide is separated and essentially free of other polypeptides, i.e. the polypeptide is the primary and active constituent.

Alternatively, a polypeptide of the invention can be isolated by cloning a nucleic acid encoding it, expressing the recombinant nucleic acid in a suitable host cell, and isolating/purifying the expressed polypeptide to remove it from other constituents of the cell. Methods of making recombinant DNAs, expressing them, and isolating/purifying the expressed polypeptides. are conventional. Some such methods are described herein. Alternatively, parts or all of an HBP molecule, or an active fragment or variant thereof, can be produced synthetically, using conventional procedures.

Recombinant or synthetically produced molecules can be used in methods of the invention. It has been shown by the present inventors that recombinant molecules are able to stimulate IL-10 production, and thus can be used to inhibit Th-1- or Th-2-associated conditions by virtue of inhibiting upregulated IL-10 in those conditions. Furthermore, recombinant molecules can attenuate reactive oxygen species (ROS), which are known to play a key role in the microvascular dysfunction at mucosal surfaces, which causes significant pathology in Th-1-associated conditions. Without wishing to be bound by any particular mechanism, it is suggested that this antioxidant activity of HBP may function independently of cytokine modulation in inhibiting Th-1-associated conditions.

HBP or an active fragment or variant thereof can be used to suppress a variety of types of inflammation. For example, the inventors demonstrate herein that HBP can activate bone marrow derived dendritic cells and alter cytokine production (e.g., increase the production of IL-10, decrease the production of IL-12, etc.) in these cells in a pattern consistent with the inhibition of Th-1- and Th-2-associated conditions. A “Th-1-associated” disease or condition, as used herein, is one that is characterized by an upregulation of the cytokines IL-2, IL-12, IFNγ and TNFβ. IL-10 can also be upregulated in Th-1 responses as a negative feedback mechanism to prevent further damage from a Th-1-associated cytokine storm. A “Th-2-associated” disease or condition, as used herein, is one that is characterized by an upregulation of the cytokines IL-10, IL-4, IL-5 and IL-13. The Th-2-associated condition, allergic inflammation, is also characterized by an IgE component. Without wishing to be bound by any particular mechanism, it is suggested that by upregulating IL-10, HBP suppresses Th-1- or Th-2-associated conditions by reducing cellular upregulation of IL-10 by a feedback mechanism. By contrast, it is suggested that by reducing the level of IL-2, HBP inhibits Th-1-associated conditions in which IL-12 is upregulated.

Furthermore, the inventors demonstrate an antioxidant activity of HBP, which supports the conclusion that HBP can be used to inhibit Th-1- or Th-2-associated conditions, and particularly indicates that HBP can be used to treat, e.g., an intestinal inflammation such as such as Crohn's disease (CD) or ulcerative colitis (UC) (either Th-2- or Th-1-associated). In addition, this antioxidant effect suggests that HBP, or an active fragment or variant thereof, can be used to treat age-related disorders (conditions or diseases), such as various types of dementia. Oxidative stress can induce changes in brain activity leading to cognitive impairment and reduced learning and memory function. The ability of HBP to act as a powerful antioxidant would be expected to reverse such disorders. The antioxidant effect of HBP on neurological effects can be demonstrated, e.g., on neuronal cells in vitro, using methods similar to those used by some of the present inventors for studying the antioxidant effects of polyphenols from cinnamon and green tea (Panickar et al. (2007) Soc Neurosci, Abs; and Panickar et al. (2008) FASEB J 22:#700.8).

The increase in IL-10 induction by HBP would also be expected to contribute to reduced proinflammatory effects due to obesity and reduced insulin function in the brain.

The findings presented herein also indicate that HBP can be used as a TLR-4 adjuvant. An adjuvant of the invention, when administered together with antigens, forms an adjuvanted vaccine or immunotherapeutic that can be delivered by a mucosal route (such as nasal, oral, oropharyngeal, ocular, geniturinary mucosal including vaginal, sublingual, intrapulmonary, intratracheal or rectal) or a parenteral route (such as intramuscular, subcutaneous, intravenous, intraperitoneal, submucosal, intradermal) or a transdermal, topical or transmucosal route to induce enhanced levels of serum and/or mucosal antibodies and/or type 1 cellular immune responses against the antigen compared with the antigen alone given by the same routes.

As noted elsewhere herein, an adjuvant of the invention can be used to enhance immunological responses against an infectious disease organism. In addition, the ability of an adjuvant of the invention to produce type 1 immune responses against an antigen will be beneficial for producing effective therapeutic vaccines, for example against cancer or autoimmune diseases where CTL and Th1 cytokine responses are important, or Th-2 polarized diseases or conditions such as allergy where a strong Th-1 response could provide negative regulation. For example, allergic rhinitis can often be effectively controlled by immunotherapy—a series of injections with increasing doses of the substance against which the individual is allergic.

It is noted that the instant invention can readily adjuvant vaccines containing single, monovalent or multi-component antigens such as peptides, proteins, toxoids, glycoproteins, glycolipids, carbohydrates and/or polysaccharides, isolated from biologic organisms of the animal or plant kingdom that may be infectious organisms, such as parasites, viruses and bacteria, or may be extracts or purified or chemically modified extracts of allergens derived from unicellular or multicellular organisms or may be chemical material. It is also envisioned that whole or disrupted microorganisms including viruses, bacteria or parasites, attenuated or inactivated could be used as antigen. These materials may also be produced by synthetic or recombinant molecular procedures to induce immunity to and protect against several strains of a particular organism or multiple organisms or disease-causing agents or against allergies, cancer or auto-immune diseases. The utility in human and veterinary fields is proposed. Furthermore, the invention can be used to enhance immunity when given together with the antigen as an adjuvanted vaccine or immunotherapeutic, as priming or boosting immunizations prior to or subsequent to administering the antigen (by mucosal or parenteral routes) without an adjuvant of the instant invention.

For parenteral, nasal, oral or suppository use, the adjuvant may be given together with amounts of a variety of pharmaceutically acceptable carriers (excipients) or other adjuvants including oils, emulsions, nano-emulsions, fats, waxes, buffers, or sugars, as diluents or vehicles customary in the art to provide stable delivery of the product in the desired delivery format.

A number of considerations are generally taken into account in designing delivery systems, routes of administration, and formulations for protein and peptide therapeutic agents, such as the polypeptides of the invention. Such therapeutic agents can include peptides that act as suppressors of allergic reactions, Th-1 mediated conditions, or Th-2 mediated conditions, or that serve as adjuvants or freestanding immunomoldulators. See, e.g., Eppstein (1988), CRC Crit. Rev. Therapeutic Drug Carrier Systems 5, 99-139; Siddiqui et al. (1987), CRC Crit. Rev. Therapeutic Drug Carrier Systems 3, 195-208, 1987); Banga et al. (1988), Int. J. Pharmaceutics 48, 15-50; Sanders (1990), Eur. J. Drug Metab. Pharmacokinetics 15, 95-102; and Verhoef (1990), Eur. J. Drug Metab. Pharmaco-kinetics 15, 83-93. The appropriate delivery system for a given polypeptide or conjugate of the invention will depend upon its particular nature, the particular clinical application, and the site of drug action.

A pharmaceutical composition of the invention generally comprises a carrier, such as a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier is selected to minimize any degradation of the active ingredient and to minimize any adverse side effects in the subject, as would be well known to one of skill in the art. For a discussion of pharmaceutically acceptable carriers and other components of pharmaceutical compositions, see, e.g., Remington's Pharmaceutical Sciences, 18th ed., Mack Publishing Company, 1990.

One skilled in the art will appreciate that a suitable or appropriate formulation can be selected, adapted or developed based upon the particular application at hand.

Formulations suitable for oral administration can consist of liquid solutions, such as an effective amount of the compound dissolved in diluents, such as water, saline, or fruit juice; capsules, sachets or tablets, each containing a predetermined amount of the active ingredient, as solid, granules or freeze-dried cells; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers. Suitable formulations for oral delivery can also be incorporated into synthetic and natural polymeric microspheres, or other means to protect the agents of the present invention from degradation within the gastrointestinal tract (see, for example, Wallace et al. (1993), Science 260, 912-915).

The polypeptides of the invention, alone or in combination with other antiinflammatory agents or with suitable antigens, can be made into aerosol formulations to be administered via inhalation. These aerosol formulations can be placed into pressurized acceptable propellants, such as dichlorodifluoromethane, propane, nitrogen and the like.

The polypeptides of the invention, alone or in combination with other anti-inflammatory agents or with suitable antigens, can be made into suitable formulations for transdermal application and absorption (Wallace et al., 1993, supra). Transdermal electroporation or iontophoresis also can be used to promote and/or control the systemic delivery of the compounds and/or compositions of the present invention through the skin (e.g., see Theiss et al. (1991), Meth. Find. Exp. Clin. Pharmacol. 13, 353-359).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; mouthwashes comprising the active ingredient in a suitable liquid carrier; or creams, emulsions, suspensions, solutions, gels, creams, pastes, foams, lubricants, sprays, suppositories, pessaries, tampons or the like.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The dose of an agent of the invention, or composition thereof, administered to an animal, particularly a human, in the context of the present invention should be sufficient to effect at least a therapeutic response in the individual over a reasonable time frame (e.g., an anti-inflammatory effective amount, an adjuvant-effective amount, etc.). The exact amount of the dose will vary from subject to subject, depending on the species, age, weight and general condition of the subject, the severity or mechanism of any disorder being treated, the particular agent or vehicle used, its mode of administration and the like. The dose used to achieve a desired antiinflammatory concentration in vivo will be determined by the potency of the particular agent employed, the pharmacodynamics associated with the agent in the host, the severity of the disease state of infected individuals, as well as, in the case of systemic administration, the body weight and age of the individual. The size of the dose also will be determined by the existence of any adverse side effects that may accompany the particular inhibitory agent, or composition thereof, employed. It is generally desirable, whenever possible, to keep adverse side effects to a minimum.

Dosages for administration of a polypeptide of the invention can be in unit dosage form, such as a tablet or capsule. The term “unit dosage form” as used herein refers to physically discrete units suitable as unitary dosages for human and animal subjects, each unit containing a predetermined quantity of a polypeptide of the invention, alone or in combination with other antiinflammatory agents, calculated in an amount sufficient to produce the desired effect in association with a pharmaceutically acceptable diluent, carrier, or vehicle.

The specifications for the unit dosage forms of the present invention depend on the particular polypeptide of the invention, or composition thereof, employed and the effect to be achieved, as well as the pharmacodynamics associated with each polypeptide, or composition thereof, in the host. In some embodiments, the dose administered is an “anti-inflammatory effective amount,” “an adjuvant-effective amount,” etc.

One skilled in the art can easily determine the appropriate dose, schedule, and method of administration for the exact formulation of the composition being used, in order to achieve the desired “effective concentration” in the individual patient.

One embodiment of the invention is a kit useful for any of the methods disclosed herein; such a kit can comprise one or more isolated polypeptides of the invention. For example, a kit suitable for a therapeutic treatment in a subject may further comprise a pharmaceutically acceptable carrier and, optionally, a container or packaging material. Among other uses, kits of the invention can be used in experiments to study mechanisms by which HBP suppresses inflammatory reactions, enhances immunological responses to an antigen, etc. A skilled worker will recognize components of kits suitable for carrying out any of the methods of the invention.

Optionally, the kits comprise instructions for performing the method. Kits of the invention may further comprise a support or matrix to which polypeptides of the invention can be attached or immobilized. Other optional elements of a kit of the invention include suitable buffers, pharmaceutically acceptable carriers, or the like, containers, or packaging materials. The reagents of the kit may be in containers in which the reagents are stable, e.g., in lyophilized form or stabilized liquids. The reagents may also be in single use form, e.g., in single dosage form for use as therapeutics, or in single reaction form for diagnostic use.

In the foregoing and in the following examples, all temperatures are set forth in uncorrected degrees Celsius; and, unless otherwise indicated, all parts and percentages are by weight.

### Examples

### I. Materials and Methods

A. Animals and Parasite Inoculation

Female or male C57BL/6 were obtained from Taconic Animal Laboratory (Rockville, Md.). All mice were housed under specific pathogen-free conditions in a Comparative Medicine Branch facility at the National Institutes of Health in an American Association for the Accreditation of Laboratory Animal Care-approved facility. The NIAID animal care and use committee approved all experimental procedures.

The acquisition and preparation of infective A. suum eggs, oral inoculation, and management of pigs were as previously described (Urban et al. (1985) Exp. Parasitology 60, 245-254). All procedures were approved by the Beltsville Animal Care and Use Committee as Protocol #07011.

B. Antibodies and Reagents

Rabbit polyclonal antibodies were made against Domain1 of AsHb from Spring Valley Laboratories, Inc. (Sykesville, Md.). Polymyxin B was obtained from Bio-Rad Laboratories (Hercules, Calif.). The Limulus Amebocyte Assay was obtained from Charles River Endosafe (Charleston, S.C.). Endoglycosidases PNGaseF and endoH from NewEngland BioLabs (Ipswich, Mass.) and endoD from US biologicals (Swampscott, Mass.). Superose 6 gel filtration column (XK 16/70 prep grade), Superdex-200 gel filtration column (XK 16/70 prep grade) and anion exchange column (Q HP hitrap) from GE healthcare Biosciences (Piscataway, N.J.). AlexaFluor 594-conjugated transferrin and TexasRed-conjugated dextran (70,000 MW) and species specific AlexaFluor conjugated secondary antibodies were obtained from Molecular Probes (Carlsbad, Calif.). Other materials and buffers were obtained from Sigma-Aldrich (St. Louis, Mo.) unless otherwise stated.

C. Preparation of Parasite Tissue Extracts

Ascaris suum PCF was obtained from adult worms recovered from intestines of naturally-infected pigs obtained from the Beltsville Agricultural Research Center USDA abattoir. The adult worms were rinsed extensively in PBS, the posterior end of the parasite was nicked, and fluid from the pseudo-coelom was aseptically collected and centrifuged at 12,000×g to remove any particulate material (McConchie et al. (2006) Infect Immun 74, 6632-6641) For A. suum muscle, intestinal, and cuticular layers, individual body tissues were dissected out, homogenized in PBS, and centrifuged at 12,000×g for 20 min to obtain clear supernatants. Excretory/secretory products from adult and L3/L4 were prepared as previously described (Rhoads et al. (1997) J Parasitol 83, 780-784). All helminth products were stored at −80° C. until use.

D. Purification of Native Ascaris Antigens

Native AsHb was purified from PCF as described earlier with the minor modifications (De Baere et al. (1992) (supra)). In brief, a Superose 6 gel filtration column (GE healthcare Biosciences, Piscataway, N.J.) was equilibrated with PBS and approximately 50 mg of PCF was applied at a flow rate of 1 ml⁻¹. Eluted proteins were collected and fractions corresponding to native AsHb were dialyzed against buffer A (20 mM Tris-HCL pH 8, 0.5 mM EDTA, 1 mM DTT and 50 mM NaCl). Dialyzed proteins were further purified to homogeneity using an anion exchange column equilibrated with the buffer A. Loaded proteins were eluted with a salt gradient of 0.05 to 1M NaCl, and fractions corresponding to AsHb were pooled and dialyzed against PBS. For the protein preparations, endotoxin free buffers and water were used to minimize LPS contamination. To remove any endotoxin associated with these protein preparations, purified proteins were passed through 10 ml of Affi-prep polymyxin B matrix (Bio-Rad) packed in a gravity column equilibrated with 10 mM phosphate buffer pH 6.5 and 100 mM NaCl. Protein preparations were tested for endotoxin using Limulus Amebocyte Assay (Charles River Endosafe, Charleston, S.C.) and the final concentration of endotoxin was determined to be less then 0.001 ng/μg of protein. In preliminary tests, this dose of LPS does not cause any BMDC activation as characterized by either a change in surface marker expression and/or cytokine production after exposure.

E. Purification of Recombinant Ascaris Antigens

Bacterial clones for recombinant full-length AsHb, heme-binding Domain1 (N-terminus) and Domain 2 (C-terminus) were prepared as described elsewhere (Kloek et al. (1993) J Biol Chem 268, 17669-17671). These clones were obtained as a generous gift from Dr. Daniel Goldberg (Washington University, St. Louis, USA) and recombinant proteins were prepared using E. coli BL21 (DE3) cells (Yang et al. (1995) Proc Natl Acad Sci USA 92, 4224-4228). In brief, bacteria expressing AsHb clones were grown at room temperature in LB media containing 100, 40 μg/ml of ampicillin and hemin (Sigma-Aldrich, St. Louis, Mo.), respectively. When growth reached log phase (O.D≅0.8), protein production was induced with 1 mM IPTG (Sigma-Aldrich, St. Louis, Mo.) and cells were harvested after 5 h by centrifugation at 3,000×g for 10 minutes. Proteins were purified from cell pellets after freeze thawing three times and were suspended in 20 ml of buffer A. Cells were lysed by sonication and centrifuged at 12,000×g for 20 min to remove cell debris. Soluble cell supernatants were filtered through 0.4 micron filters and applied on pre-packed 5 ml anion exchange column (GE healthcare Biosciences) equilibrated with buffer A. Bound proteins were eluted using a salt gradient from 0.05 to 1 M NaCl. Peaks corresponding to AsHb were detected by absorption at 410 nm, were pooled and concentrated using 10 kDa cutoff Amicon Ultra-4 centrifugal concentrators (Millipore, Billerica, Mass.). Concentrated proteins were further purified to homogeneity by passing through Superdex-200 gel filtration column (GE healthcare Biosciences) equilibrated with PBS. Homogeneity of purified proteins was more then 98% as confirmed by running on 12% SDS-PAGE under reducing conditions. To remove endotoxin in these protein preparations, purified proteins were passed through 10 ml of Affi-prep polymyxin B matrix (Bio-Rad, Hercules, Calif.) packed in a gravity column equilibrated with 10 mM phosphate buffer pH 6 and 100 mM NaCl and tested for LPS as described above. The final amount of endotoxin in recombinant protein preparations was less then 0.001 ng/μg of protein. Finally, purified proteins were dialyzed against PBS and protein was estimated using BCA method (Pierce, Rockford, Ill.).

F. Preparation of Dendritic Cells from Bone Marrow (BMDC):

BMDC were generated using a method described earlier with minor modifications (De Baere et al. (1992) (supra)). In brief, bone marrow cells collected by flushing tibia and femur with RPMI 1640 medium (Gibco Life Sciences, NY) and particulate material removed by passing through 70 μM nylon mesh. RBCs lysed using ACK lysis buffer and cells were washed twice with RPMI medium supplemented with 10% fetal bovine serum, 10 mM HEPES, 50 uM β-mercaptoethanol, 10 μg/ml gentamycin and recombinant mouse GM-CSF (40 ng/ml; PeproTech, Rocky Hill, N.J.). The cells were incubated at 37° C. in 5% CO₂ atmosphere and on days 2 and 5, media was replenished. On day 7, non-adherent BMDC were harvested and analyzed by FACS for the surface expression of CD11c (Clone, HL3), CD40 (Clone, HM40-3), CD86 (Clone, GL1) and MHCII (Clone, AF6-120.1) (BD Biosciences, San Diego, Calif.). Cultured cells were comprised of greater than 90% of CD11c⁺ cells with a lower expression of activation markers resembling an immature DC phenotype. For specific BMDC treatments, cells were seeded in low adherence 24 well plates at 0.5 million cells per ml. After resting overnight, BMDC were exposed to native (10 μg/ml) or recombinant AsHb (0.5 μM) preparations for 12 h, or were co-stimulated with LPS (50 ng/ml) for 12 h.

G. Isolation of Pig BAL

Bronchial-alveolar lavage cells were obtained from the lungs of pigs infected with either A. suum or uninfected control pigs after euthanasia with an overdose of Euthasol (Virbac Animal Health, Fort Worth, Tex.) (Huelsenbeck et al. (2001) Bioinformatics 17, 754-755). Briefly, the large right lobe of the lung was gravity-filled with 200-250 ml of PBS, followed by massage for 30 seconds and draining of the cell suspension into 50 ml polypropylene tubes. The cells were washed and re-suspended in RPMI 1640 medium with 5% heat-inactivated fetal bovine serum, and cell counts and viability were determined after Trypan blue staining. Bronchial-alveolar lavage cells were identified with specific monoclonal antibodies (mAb) against porcine macrophages (SWC9) (Clone PM18-7) (Serotec, Raleigh, N.C.) and against porcine granulocytes (SWC3) (Hybridoma 74-22-15) kindly provided by Dr. Joan Lunney, ARS, USDA, Beltsville, USA) and IgG1 and IgG2b isotype controls (Serotec, Raleigh, N.C., USA).

H. Induction of Alveolar Macrophage Extra Cellular Reductants

Staphylococcus aureus S5, 8, and 336 bacteria were kindly supplied by Dr. Max Paape from the Bovine Functional Genomics Laboratory, ARS-USDA, Beltsville, Md. An aliquot of 1×10⁸ S. aureus bacteria was opsonized after incubation with 200 μl of hyper immune pig serum under constant rotation using a Rotamix (ATR Inc, Laurel, Md.) for 1 h at room temperature. Opsonized bacteria were added to 2×10⁶ pig BAL cells, which were then incubated for 30 minutes with constant rotation at 4° C., and centrifuged at ×800 g for 10 minutes. The cell concentration was adjusted to 5×10⁵ cells/ml with HBSS before setting the assay in a 96-well flat bottom micro-plate. Super oxide production was measured in triplicate. Half of the wells were given 20 μl super oxide dismutase (Sigma, 3000 units, 1 mg/ml in HBSS) before adding cells at 100 μl/well. The volumes of the wells were adjusted with HBSS. Fifty μl cytochrome C (Sigma, 4.2 mg/ml in HBSS) was added to all wells and the plate incubated at 37 C. The optical density (OD) at 550 nm was measured after 30, 60, 90, and 120 min (Solano et al. (1998) Can J Vet Res 62, 251-256).

I. Measurement of Cytokines

Pro- and anti-inflammatory cytokines were measured using Lincoplex kit for mouse cytokine multiplex Immunoassay (Linco Research Inc., St. Charles, Mo.) per manufacturer's instructions. BMDC culture supernatants from each treatment were assayed undiluted in triplicate by incubating with Antibody-Immobilized microbeads overnight (16-18 h) at 4° C. After washing, the samples were then incubated with Detection Antibody Cocktail for an hour before addition of Streptavidin-Phycoerythrin. Median Fluorescence units in the filter plate was measured by counting 50 beads per bead set in 50 μl sample size using the Liquichip reader (Qiagen, Valencia, Calif.).

J. Immunoblotting and Immunohistochemistry

Ascaris suum PCF antigens including AsHb (5 μg/lane) were separated by one or two-dimensional gel electrophoresis and transferred onto nitrocellulose membranes (Millipore). The membranes were blocked by incubating for 30 min with 5% skimmed milk. For the detection of the antigenic proteins, membranes were incubated with the serum (1:3000) obtained from pigs inoculated with 10,000 infective A. suum eggs, rested for two weeks, and re-challenged followed by a bleed two weeks later. For the detection of AsHb, membranes were incubated with rabbit anti-AsHb domain 1 polyclonal antibodies (1:10000). Pre-immune rabbit sera were used as a negative control. Membranes were incubated with corresponding primary antibodies for 1 hr and extensively washed with PBS containing 0.1% Tween 20 before incubating with peroxidase-conjugated goat anti-pig or rabbit IgG (Sigma-Aldrich, St. Louis, Mo.) as a secondary antibody. After the membranes were washed, the proteins bound to the secondary antibody were visualized with DAB substrate kit (BD Biosciences, San Diego, Calif.).

For immune histochemical studies, pigs were inoculated with 10,000 infective A. suum eggs. Larvae were harvested on day 7 from the lungs, and days 10 and 14 from the intestines (Morimoto et al. (2003) Exp Parasitol 104, 113-121). Harvested larvae were immediately placed in 10% neutral buffered formalin and embedded in paraffin. Tissue sections were treated with antibodies to AsHb by the ABC method (Vector Laboratories, Burlingame, Calif.). Slides were examined under a microscope and images were analyzed using Adobe Photoshop version 7.0 (Adobe, San Jose, Calif.)

K. Ascaris Hemoglobin Specific ELISA

The ELISA plates (Nunc, Rochester, N.Y.) were coated overnight at 4° C. with 0.5 μg/well of native AsHb in carbonate buffer pH 9.6. The following day, plates were rinsed with wash buffer (0.05% tween-20 in PBS) and unbound sites were blocked by treatment with blocking buffer (10% FCS in PBS) for 2 h. The sera from mice infected with A. suum or PBS control group were diluted in blocking buffers, dispensed in triplicate and incubated overnight at 4° C. After washing, the corresponding biotin conjugated anti-IgG subclass monoclonal antibodies (BD Pharmingen, San Diego, Calif.) were added 100 μl/well at 1:250 dilution in blocking buffer and incubated for 2 h at room temperature. Wells were washed and incubated with 100 μl of avidin-HRP (1:250) for 1 h before the color was developed using 100 μl of TMB substrate solution (eBiosciences, San Diego, Calif.). The reaction was stopped after 2 min with 50 μl of 2M sulfuric acid and absorbance read at 450 nm. Wells without serum were used as controls to subtract background color.

L. Confocal Microscopy

One million mouse BMDCs or pig BAL cells were cultured for 30 minutes on poly-L-lysine coated sterile coverslips in 12-well tissue culture plates with 1 ml of RPMI 1640 media (without phenol red). Cells were incubated with AsHb (25 μg/ml) and AlexFluor-594 conjugated transferrin or -dextran 70,000 Da (100 μg/ml) for 30 min. After incubation, cells were washed with PBS to remove free antigens, fixed in 4% paraformaldehyde for 15 minutes, then permeabilized in 0.1% Triton X-100. The fixed and permeabilized cells were blocked in a blocking buffer (1% BSA in PBS) and stained for 30 minutes with primary rabbit polyconal antibodies to AsHb diluted (1:250) in a blocking buffer. Unbound antibodies were removed by washing three times with 0.1% Triton X-100 in PBS and stained for 30 minutes at room temperature with AlexFluor-488 or 594-conjugated secondary Abs (Molecular Probes, Carlsbad, Calif.) diluted (1:1000) in a blocking buffer. Confocal microscopy was performed with a ×63 oil-immersion lens in the sequential mode using TCS SP2 AOBS microscope (Leica) and images were analyzed using Imaris (version 4.2.0; Bitplane) and Adobe Photoshop (version 7.0)

### II. AsHb Activates Bone Marrow Derived Dendritic Cells

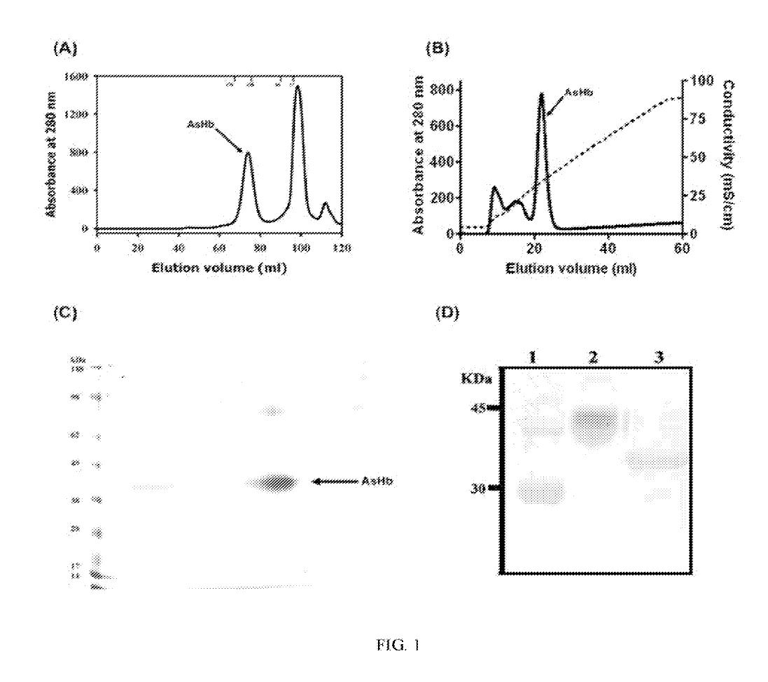

We have previously demonstrated that PCF modulates dendritic cell function in response to LPS. In addition, PCF suppresses the production of the inflammatory cytokines IL-12 and TNF-α while augmenting IL-10 production in a dose response manner. To delineate the possible immune modulating proteins present in PCF, the fluid was separated into different protein fractions based on their size using a gel filtration column (Superose 6). This separation resulted into two major components, a high molecular weight (HMW) fraction and a low molecular weight (LMW) fraction corresponding to the molecular sizes ˜300 kDa and ≦50 kDa, respectively (FIG. 1A). These fractions were evaluated for their ability to either activate or alter cytokine responses in bone marrow-derived dendritic cells (BMDCs). When compared to media alone, exposure to the HMW fraction altered the cytokine production by BMDC in response to LPS. This effect is associated with the HMW fraction but not by the LMW fraction. The HMW fraction was further purified using anion exchange chromatography, and the major protein peak associated with the modulation of BMDC mediated cytokine production was purified to homogeneity (FIGS. 1B and 1C). The purified protein was separated using 2D gel electrophoresis and was determined to have an apparent molecular mass of 42 kDa (FIG. 1C). Mass spectrometry was performed on the excised band, and the derived peptide sequences were compared with translated sequences from the A. suum Expresses Sequence Tag database (A. suum EST) (Steen et al. (2004) Nat Rev Mol Cell Biol 5, 699-711). Data from several tryptic peptide sequences indicated the 42 kDa was an extracellular hemoglobin. FIG. 1D shows the detection of AsHb as an immunogenic protein by probing with serum derived from A. suum infected pigs. The higher molecular weight of native AsHb (Lane, 2) compared with recombinant AsHb (lane, 3) supports the possibility of glycosylation in AsHb (FIG. 1D).

### III. AsHb Alters Cytokine Production in BMDC

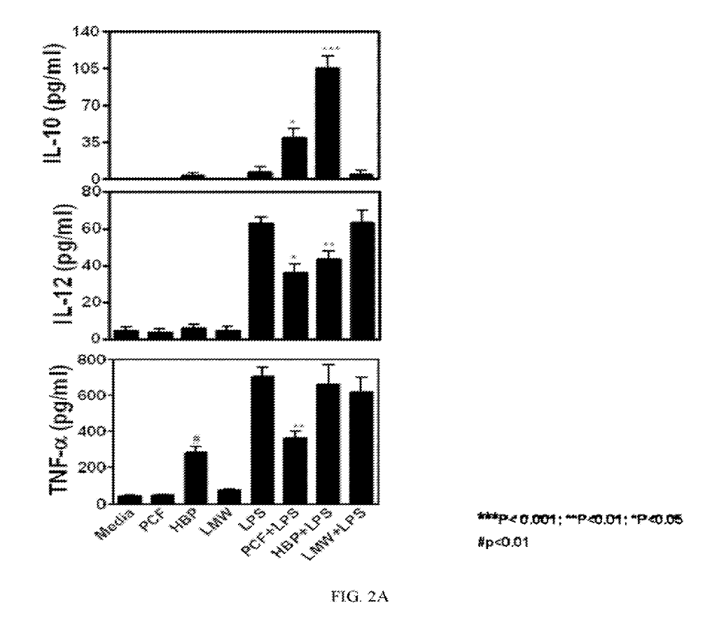

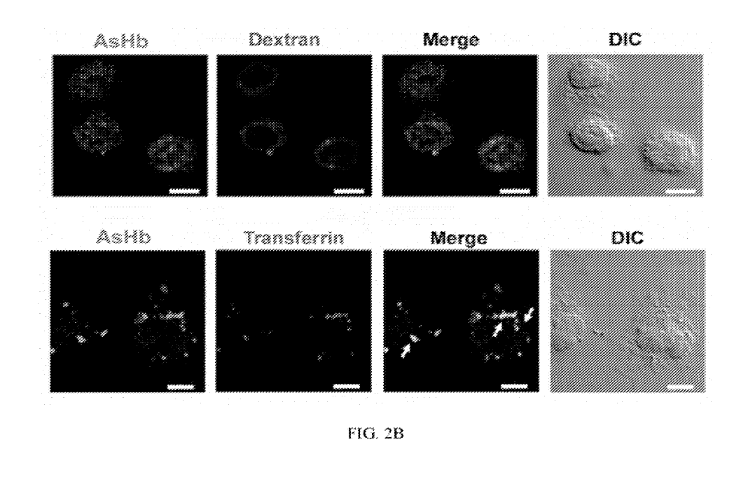

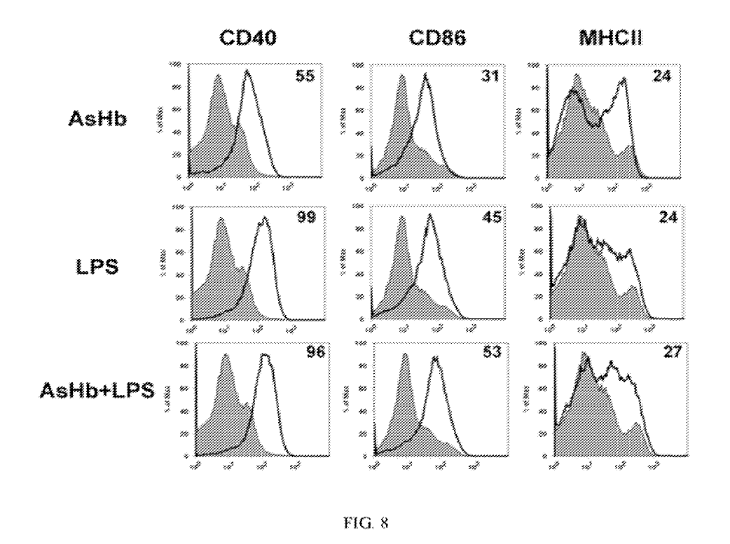

The purified AsHb-mediated activation of BMDC was evaluated by assessment of surface changes in activation markers and cytokines along with the controls, including PCF and the low molecular weight protein fraction. Stimulation of BMDC with PCF or the LMW fraction had no effect on TNF-α, IL-12 or IL-10 (FIG. 2A) compared with media alone. Exposure of BMDC to AsHb induced the production of TNF-α (p<0.01; FIG. 2A) but had no effect on the production of IL-10 or IL-12. Co-stimulation of BMDC with PCF and LPS inhibited the production of Th1 pro-inflammatory cytokines IL-12 and TNF-α (p<0.01) and significantly increased the production of IL-10 (p<0.05; FIG. 2A). Conversely, co-stimulation of BMDC with AsHb and LPS induced IL-10 (p<0.001) and reduced IL-12 (p<0.05). Compared with PCF, AsHb demonstrated 10 times greater activity in the induction of IL-10 from LPS-treated BMDC. This finding suggests that AsHb may be the component present in PCF that stimulates IL-10 production and reduces IL-12 production in LPS-co-stimulated BMDC (FIG. 2A). The additional ability of PCF to inhibit LPS induced TNF-α production (p<0.01) suggests that other components present in PCF may be involved in the regulation of TNF-α production. Immature BMDCs exhibit low levels of surface expression of CD40, CD86, and MHC II. Exposure of BMDC to the LMW protein fraction did not effect the expression of surface activation markers or cytokine production when compared with untreated BMDC. Interestingly, treatment of BMDC with AsHb alone (10 μg/ml) increased the expression of surface activation markers CD40, CD86, and MHC II (FIG. 8). However, co-stimulation of BMDC with AsHb and LPS showed no alterations in the surface activation marker expression (FIG. 8).

To examine whether dendritic cells internalize AsHb, we examined the interaction of AsHb with BMDC using confocal microscopy. BMDC were incubated with AsHb (25 μg/ml) and then fixed, permeabilized, and stained with antibodies to AsHb. A fluorescence signal for AsHb was found on both the surface and intracellular compartments of BMDC (FIG. 2B). To study the mechanism of internalization, BMDC were incubated with AsHb and fluorescently-conjugated transferrin or dextran (70 kDa). Transferrin is a marker for receptor-mediated endocytosis and dextran is a marker for fluid phase endocytosis or phagocytosis. As shown in FIG. 2B, AsHb, transferrin, and dextran internalized into the intracellular compartments of BMDC. However, there was no co-localization observed between AsHb and dextran (e.g., as shown in FIG. 2B). Conversely, both transferrin and AsHb co-localize and exist in the same intracellular compartments, suggesting that AsHb is internalized through a receptor mediated endocytic pathway (e.g., as shown in FIG. 2B). To substantiate our mouse model findings with the natural host of A. suum, we evaluated the uptake of AsHb in swine BAL cells and intestinal epithelial cells. Similar to mouse BMDC, localization of AsHb was observed with transferrin but not dextran.

### IV. Heme-Binding Globin-Like Domains in AsHb

Ascaris hemoglobin contains 338 amino acids with a total calculated mass of 40.6 kDa and apparent molecular mass of about 45 kDa due to the presence of two N-glycosylation sites (FIGS. 1C and 3B). Unlike mammalian hemoglobins, AsHb is an oligomeric protein of eight subunits corresponding to ˜320 kDa. Ascaris hemoglobin is predominantly an α-helical protein with two globin-like domains (FIG. 3A). Each domain binds a single heme molecule resulting in two hemes/subunit (Darawshe et al. (1991) Eur J Biochem 201, 169-173). To understand the conservation of globin-like domains present in AsHb within nematodes, we have aligned heme-binding globin-like domains of A. suum with parasitic and non-parasitic nematode sequences present in the EST databases of nematodes. Alignment of the sequences between domain 1 and domain 2 demonstrate a 62% sequence identity with significant differences at their C-terminal ends (FIG. 3B). Several amino acid positions (B10, B4, C2, CD1, E7 and F8) are conserved that play critical roles in heme binding (FIG. 3B). Most notable, glutamine at E7 position contributes to the high oxygen affinity in Ascaris hemoglobin (Yang et al. (1995) (supra)). Most of these residues are invariant among nematode sequences, with the notable exception of position EF3 which is not conserved in free-living nematodes (FIG. 3B). As shown in FIG. 3B, the presences of N-glycosylation sites are also restricted to parasitic nematodes. The absence of N-glycosylation sites in free-living nematodes such as C. elegans suggests that AsHb may have a role in the evolution of Ascarids as parasitic worms, as AsHb may regulate internal free oxygen in the pseudo-coelom and produce a secreted product that can capture host-generated free radicals. In addition, Domain 2 also contains a hydrophilic C-terminal tail that may play a critical role in the oligomerization of AsHb (FIGS. 3A and 3B).

A Bayesian phylogenitic analysis (Huelsenbeck et al. (2001) (supra)) was performed between different nematode AsHb sequences to evaluate the evolutionary relationship of the globin domains (FIG. 3C). As shown in FIG. 3C, the phylogenetic tree indicates a duplication event that may have led to the generation the dimeric globin in the parasitic nematodes. Results show that the relationship among domain 1 sequences is not well resolved. In contrast, domain 2 sequences from A. suum and Pseudoterranova decipiens (cod worm), unambiguously group together and may represent a derived state from domain 1 (FIG. 3C). A second globin-like domain found in the parasitic nematodes evaluated herein was not observed in free-living worm C. elegans.

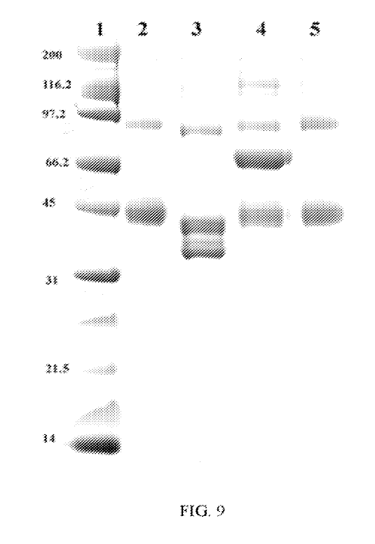

Nematodes express large variety of glycans that may have immune modulating properties. Ascaris hemoglobin is a glycoprotein with two putative N-glycosylation sites (FIG. 3B). The nature of glycans present on AsHb were evaluated using three different endoglycosidases that differ in their deglycosylation activities. Deglycosylation was monitored using a shift in the protein migration on SDS-PAGE upon treatment with endoglycosidases H, D, and PNGase F. Ascaris hemoglobin showed deglycosylation to PNGaseF but not to endoglycosidaseH or D (FIG. 9).

### V. Role of Different Domains in AsHb for IL-10 Production

Recombinant proteins for Domain 1 (rD1), Domain 2 (rD2) and full-length AsHb (rAsHb) were expressed and purified to homogeneity (FIG. 4A). As reported previously, in solution domain 1 exists as a monomer while domain 2 and full-length recombinant AsHb were able to form octamers similar to native AsHb (e.g., Kloek et al. (1993) (supra)). Endogenous LPS contamination that may associate with these recombinant protein preparations was eliminated by sequential passage through a polymyxin B matrix. The above three recombinant preparations of AsHb were studied for their ability to modify cytokine production in BMDC to LPS stimuli. Compared to LPS alone, BMDC stimulated with LPS and domain 2 or full-length recombinant AsHb increased IL-10 production (FIG. 4B). No significant differences were observed for IL-12 or TNF-α production upon stimulation with rD1, rD2 or rAsHb in the presence of LPS (FIG. 4B).

### VI. Ascaris Hemoglobin is an Antigenic and Secretory Protein

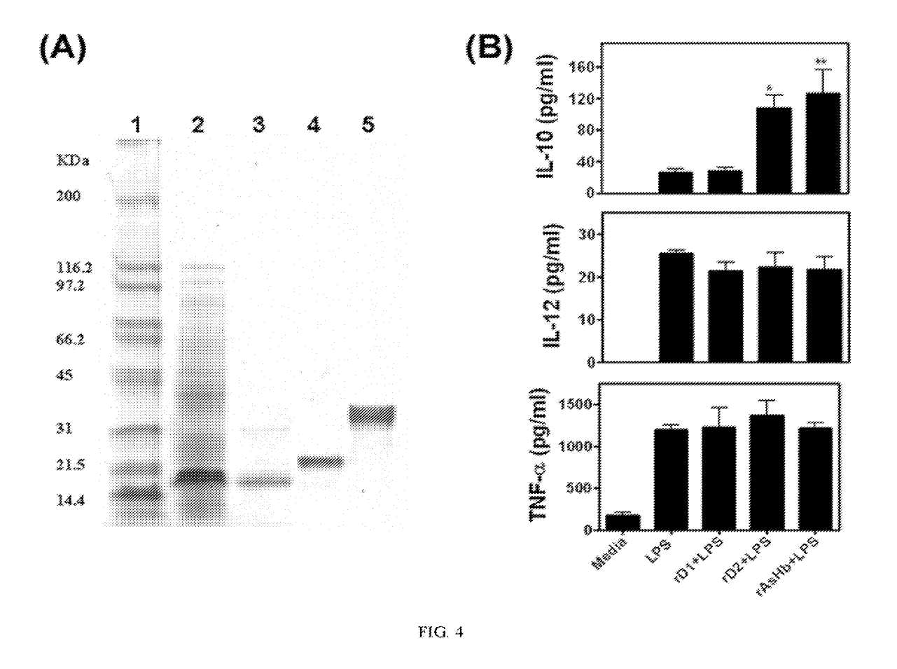

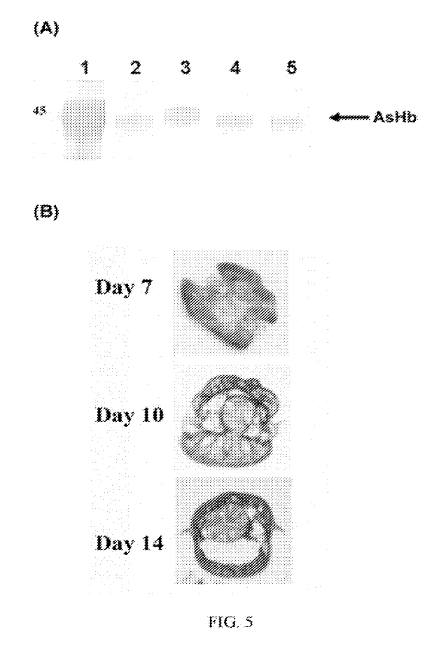

The presence of an N-terminal signal peptide in AsHb suggests that it could be processed through the Golgi and ER for glycosylation during its synthesis prior to acting as an extra cellular excretory protein. To test this hypothesis, we raised rabbit polyclonal antibodies to domain1 of AsHb and studied the expression and secretion of AsHb during parasite development. Immunoblots (FIG. 5A) showed the presence of AsHb in excretory/secretory products (ESP) prepared from both adult and L3/L4 stages. Positive binding was also observed on protein extracted from parasite muscle and intestine extracts. Antibodies to purified native and rAsHb were also observed in serum from A. suum-infected swine (FIG. 1D), suggesting that AsHb may be secreted during infection. Immunohistochemical staining of Ascaris larval stages in the lungs and intestine using AsHb specific antibodies indicated strong binding to the muscle and intestine (FIG. 5B). Antibody binding was absent in the cuticle (FIG. 5B).

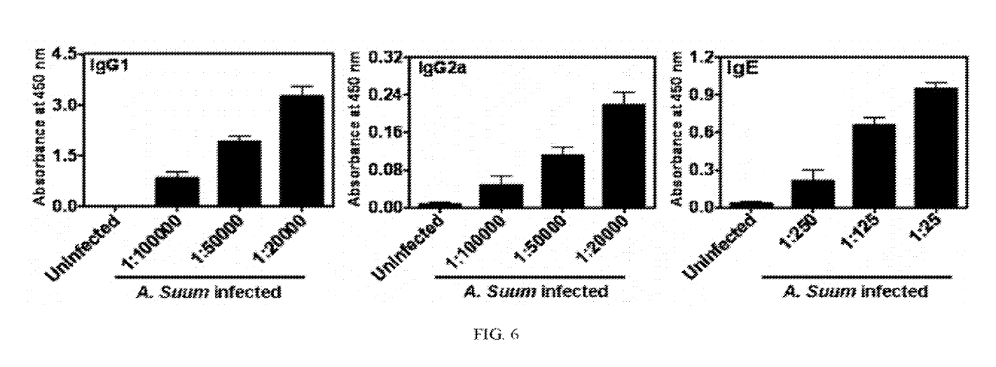

To further characterize AsHb-specific host immune responses, we used a mouse model of Ascaris infection (Schopf et al. (2005) Invest Ophthalmol Vis Sci 46, 2772-2780). Serum obtained from A/J mice infected with A. suum eggs showed very strong IgG1 response compared to IgG2a and a detectable IgE response. Development of A. suum in the mouse does not extend past the early L4 in the intestine. No antibodies to AsHb were observed in the serum from uninfected control mice (FIG. 6).

### VII. Secreted AsHb Acts as a Scavenger of Super Oxide Anion

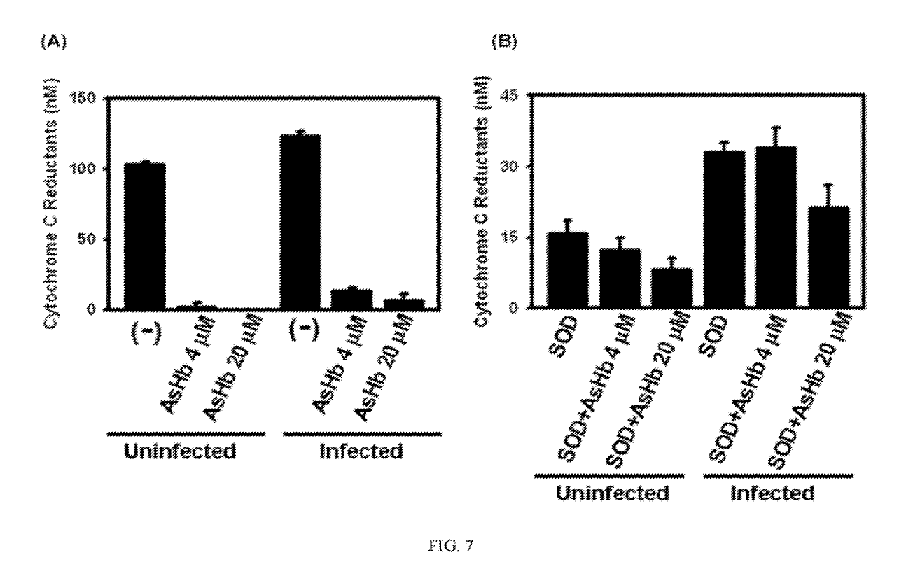

Bronchial-alveolar cells isolated from the lungs of naïve pigs have been shown to secrete super oxide anion and other free radicals when incubated with opsonized-bacteria, and the level of secretion is significantly increased when BAL cells are isolated from A. suum-infected pigs (Solano-Aguilar G I, Beshah, E., Kringel, H., Kendall, A., Restrepo, M., Ledbetter, T., Morimoto, M., Schoene, N., Zarlenga, D., Mansfield, M. S., Dawson, H. D., Urban, Jr. J. F (Submitted) Ascaris suum infection reduces phagocytic efficiency of swine alveolar macrophages). This super oxide and free radical production represents a putative host protective response against migrating larvae that are susceptible to free radical attack. The secretion of AsHb from larval and adult stages with its high avidity for oxygen suggests that AsHb can act as a molecular sink to interfere with oxygen-dependent host protective responses. To test this hypothesis, either 4 μM or 20 μM of AsHb were added to cultures of BAL cells from both naïve and Ascaris-infected pigs that were incubated with opsonized-S. aureus. The addition of AsHb reduced the level of cytochrome c reductants and non-super oxide dismutase inhibited reductants (FIG. 7).

VIII. Studies with Human Cells

Studies are performed with DC derived from human monocytes. Similar protocols are followed as in the studies described above: DC are pre-incubated with HBP or PCF followed by LPS or another TLR stimulant, and cytokine production is analyzed. The results are expected to be essentially the same as was seen in DC from mice.

IX. Studies Showing that HBP, or Domain 2 of that Protein, can Suppress Allergic Asthma in an In Vivo Mouse Model (a Th-2-Associated Condition)

We will carry out experiments by the methods described, e.g., in Schopf et al. (2005), supra; Bundoc et al. (2003), Curr Opin Allergy Clin Immunol. 3, 375-9; and McConchie et al. (2006) Infect Immun. 74, 6632-41 to confirm the following expected activities and properties of HBP in vivo: HBP significantly suppresses ragweed mediated allergic lung inflammation; HBP protects against RW-induced eosinophilic infiltration in lungs and conjunctiva; HBP treatment significantly reduces RW-induced pro-inflammatory cytokine levels in BALF; HBP treatment decreases pro-inflammatory cytokine transcript levels in whole lungs; HBP exposure downregulates splenocyte cytokine responses in an allergen-specific in vitro recall assay; and HBP exposure attenuates allergen-induced airway hyper-responsiveness.

For example, we will assess the in vivo effects of A. suum HBP in a ragweed sensitized mouse model of asthma using histological examination. IL-4, IL-5 and IL-13 cytokine levels are assessed using multiplex bead immunoassay in BAL fluid. In vitro studies using bone marrow-derived dendritic cells (BMDC) are employed to evaluate the suppression of co-stimulatory molecules such as CD40 and CD86 in response to HBP by flow cytometry. Production of Th1/Th2 cytokines by BMDCs in response to HBP exposure are assessed using a multiplex bead immunoassay procedure. Protection is expected to be evidenced by significantly decreased cell numbers in BALF, decreased eosinophilic infiltration and reduced lung pathology in an RW/HBP group compared to an RW alone group. IL-5 and IL-13 levels in BALF are expected to be significantly reduced in the HBP group compared to RW alone. HBP is expected to be able to down-regulate the expression of costimulatory molecules and the production of IL-12 by dendritic cells in response to LPS in vitro, demonstrating its immunosuppressive properties. Inactivation of proteins tend to increased BMDC activation is expected to be evidenced by upregulation of CD40 and CD86 as well as increased IL-12 production.

### X. Studies in a Porcine Model

Experiments as described in Example IX (systemic sensitization in mice with RW via intraperitoneal injection, with or without HBP, followed by RW challenge in the eye) are performed in a pig model. Conventional methods are used to scale up from a 20 g mouse to a 200 lb. pig. These studies, in a model that is particularly relevant to humans, are expected to confirm the earlier studies in mice.

XI. Studies Showing that HBP can Suppress Colitis in an In Vivo Mouse Model (a Th-2-Associated Condition)

The following studies suggest that HBP can be used to treat colitis (e.g., Crohn's disease (CD) and ulcerative colitis (UC), popularly known as inflammatory bowl disease (IBD)), as well as other Th2 driven pathologies, such as allergic conditions or diseases.

### A. Introduction and Background

Helminths have been shown to modulate mucosal immune responses orchestrated by an increase in the T regulatory cells and cytokine production by lamina propria mononuclear cells. Recent studies using excretory/secretory products from helminths have shown that they reduce cytokines IL12 and IFN-γ but induce IL10 and Th2 cytokines. These responses were shown to permit the helminths to inhibit excessive intestinal inflammation. Furthermore, preliminary studies in humans on the therapeutic use of helminthes in treating IBD are encouraging and studies have shown that helminth colonization can effectively reduce symptoms and inflammation caused by ulcerative colitis. However, helminth infection, itself, can lead to unwanted symptoms or pathology during the treatment of IBD.

The production of proinflammatory cytokines and reactive oxygen species at mucosal sites have been shown to be predominant causative agents in the pathology of IBD (as well as allergies). Acceleration of inflammation due to reactive oxygen species (ROS) can lead to microvascular dysfuction at mucosal sites. Thus altered function of endothelial cell function is thought to be responsible for the pathology of chronic IBD.

This Example suggests that the HBP component of Ascaris is beneficiary for the treatment of colitis in a mouse model. HBP is shown to attenuate ROS production and to restore microvessel function.

### B. Experimental Methods

1. Induction of Colitis

Mice (c57BL6) receive 3% DSS (45 kD; TDB Consultancy AB, Uppsala, Sweden) in the drinking water for 5 days followed by a regime of 7 or 9 days of water (reflecting acute inflammation) or 20 or 31 days of water (reflecting chronic inflammation). Control healthy mice are allowed to drink only water. Fresh DSS solutions are prepared daily and mice are recorded daily for the general health condition including, diarrhea, rectal bleeding, hunched posture and body weight. The number of mice used for each treatment is about eleven to twelve, and about three healthy mice are included in each study. At necropsy, the fecal consistency score (scale 0-3) and the inflammatory score, reflecting the degree of inflammation in the colon, based on the edema (scale 0-3), thickness (scale 0-4), stiffness (scale 0-2) and ulcerations (scale 0-1) is assessed, resulting in a total inflammatory score of 10.

2. Treatment of Colitis

Purified endotoxin free AsHb is administered during the acute phase and in the chronic phase of colitis to investigate the therapeutic effects of AsHb. In the acute phase, different doses of AsHb are administered intra-peritoneally on day 2,5 and 8 and in the chronic phase on day 13, 16, 19 after starting DSS. The acute phase study is terminated on about day 12 (5+7 d) and the chronic phase study on about day 25 (5+20 d). The respective vehicles are administered at the same time points as the drug in question.

3. Tissue and Plasma Sampling

At the end of each study period, mice are euthanized with sodium barbital and blood is collected in EDTA-containing tubes by cardiac puncture. Plasma is frozen and kept at −80° C. until analysis. The intestines are excised and carefully rinsed with saline (Gibco, Invitrogen Corp). The distal 3 cm of the colon is removed, weighed and assessed for its inflammatory score as described above. This segment is further divided longitudinally in two pieces. One piece is used for histological evaluation by rolling it as a “Swiss role”, fixing in Zinc-formalin solution (pH 7.4) and imbedding in paraffin, whereas the other piece is directly frozen in liquid nitrogen and used for cytokine and chemokine analysis as described.

4. Analysis of Local and Systemic Inflammatory Markers

Plasma levels of IL10, IL-6, IL-12p70 and TNF-alpha are analyzed by xMAP technology developed by Luminex Corporation (Austin, Tex., USA). The dissected distal colonic tissue is homogenized in PBS (Gibco), supplemented with complete mini proteinase inhibitor cocktail (Roche Molecular Biochemicals, Germany) and fetal calf serum (Gibco) as previously described. Expression of IL-1β, IL-6, IL-12p40, IL-17, IFN-γ, JE (mouse MCP-1/CCL2), RANTES/CCL5, KC/CXCL1, and MIG/CXCL9 are analyzed by ELISA (R&D Systems, UK) and by the xMAP technology (Luminex Corporation). The protein values are expressed as pg/100 mg colonic tissue.

5. Histology

Five micrometers tissue sections of distal colon (3 cm) is stained with hematoxylin/eosin (H&E). The efficacy of treatment is analyzed on H&E stained tissue using a standard microscope. A histology score reflecting infiltration of inflammatory cells and epithelial structure is established in an scale of 0 to 6, where 0=no signs of damage; 1=few inflammatory cells, no signs of epithelial degeneration; 2=mild inflammation, few signs of epithelial degeneration; 3=moderate inflammation, few epithelial ulcerations; 4=moderate to severe inflammation, ulcerations in more than 25% of the tissue section; 5=moderate to severe inflammation, large ulcerations of more than 50% of the tissue section; 6=severe inflammation and ulcerations of more than 75% of the tissue section.

### C. Results

It is expected that in the treated mice, the onset and relapse of inflammatory bowel diseases (IBD) is prevented by attenuating ROS production and inflammatory cytokines, thereby inhibiting subsequent pathological changes involved in central inflammatory pathways.

XII. Studies Showing that HBP Acts as an Anti-Inflammatory Agent for Inflammatory Bowel Disease (IBD) and Infectious Colitis, in Mouse and Pig Models (Th1 Model)

The incidence of IBD and infectious colitis in Westernized societies has been increasing over the last several decades and is generally attributed to an immunological imbalance induced by localized bacterial infection that can partially be corrected by exposure to worm parasites. In fact, therapeutic application of infectious Trichuris suis (pig whipworm) eggs to patients suffering from Crohn's disease and ulcerative colitis is currently in phase 3 trials worldwide. Without wishing to be bound by any particular mechanism, it is suggested that a localized worm infection at the site of IBD can generate immune modulation via activation of Th2-based responses to the worm that down regulate the Th1-based pathology at the disease site. This model involves a heterologous infection with parasitic eggs in a non-compatible host that generally leads to a natural abbreviation and assures a level of safety from the inability of the worm to propagate. Nevertheless, application of a worm-derived soluble product for a live infection has several advantages likely to improve safety and efficacy. Ascaris suum and crude products derived from its body wall and PCF have been also been demonstrated to have immune modulating properties in several autoimmune disease models. The use of purified and recombinant forms of HBP to an IBD and infectious colitis model would represent a reasonable application of its potential to reduce inflammation and interact with microbial products.

Testing of HBP will proceed in a two host species model of bacterial and chemically induced colitis in mice and pigs. The dose and routes of exposure of the mouse to HBP can be more effectively investigated and then scaled up to evaluate modulation of colitis in a large animal model in the pig. The infectious model in C57B1/6 mice will utilize an infection with 1.0¹⁰ cfu Citrobacter rodentium (Cr) as described by Smith and Shea-Donohue (personal communication, “Dietary changes in selenium and vitamin E affect the infectivity and inflammation from Citrobacter rodentium,” Infect. Immun. (in revision)). The level of clearance of Cr locally in the colon and systemically in the spleen and MLN will be evaluated by microbiological culturing techniques at days four, seven, 12, and 16 post-infection. Colonic pathology will be assessed histologically using H&E-stained sections. Colonic tissue and MLN will be collected and analyzed for changes in gene expression by real-time PCR. Systemic and fecal Cr-specific antibody production will be evaluated by ELISA, cellular response evaluated by cytokine production and cell surface phenotype of isolated cell populations. The treatment route, timing, dosage routes, and carrier formulation of HBP will be evaluated by standard procedures and based on details from earlier studies in the allergic models described above. Comparable application of HBP to pigs as a large animal surrogate for testing in humans and after extrapolation of conditions determined experimentally in mice will follow as described by Hontecillas et al. (2002) J Nutr. 132, 2019-27.

Acute colitis is generally evaluated in mouse models using chemical induction of inflammation by a variety of agents including exposure to dextran sodium sulfate (DSS) and TNBS. The treatment route, timing, dosage routes, and carrier formulation of HBP will be evaluated by standard procedures and based on details from earlier studies in the allergic models described above and using a model of DSS-induce colitis in mice described by Bassaganya-Riera et al. (2004) Gastroenterology 127, 777-91. Scaling up the testing of HBP to a large animal pig model of human acute colitis will follow these studies using procedures described by Bassaganya-Riera et al. (2006) Clin Nutr. 25, 454-465. For a summary description of the groups and treatments and outcomes see Table 1.

TABLE 1
HBP modulation of chemical and infection-induced colitis

| | Route of | | Vehicle for | | | Molecular and | |
|---|---|---|---|---|---|---|---|
| Group/ | HBP | Dose of HBP | delivery of | | Pathology | Immunological | |
| Species | exposure | exposure | HBP | Inducing agent | Readout | Readout | Rationale |
| Mouse | IP, ID, PO, | Variable | Aduvants, | Cr, Trichuris | H&E | Gene | Small animal |
| | SC, IV, | based on | encapsulation, | muris, etc. | | expression, | model of |
| | Ocular, etc | earlier studies | etc. | | | humoral and | infectious |
| | | | | | | cellular | colitis. |
| | | | | | | responses | |
| Mouse | IP, ID, PO, | Variable | Aduvants, | DSS, TNBS, | H&E | Gene | Small animal |
| | SC, IV, | based on | encapsulation, | etc. | | expression, | model of |
| | Ocular, etc | earlier studies | etc. | | | humoral and | chemically |
| | | | | | | cellular | induced acute |
| | | | | | | responses | colitis. |
| Pig | Optimized | Optimized | Optimized | Brachyspira | H&E | Gene | Large animal |
| | from mouse | from mouse | from mouse | hyodysenteriae, | | expression, | model of |
| | model | model | model | Trichuris suis, | | humoral and | infectious |
| | | | | etc. | | cellular | colitis. |
| | | | | | | responses | |
| Pig | Optimized | Optimized | Optimized | DSS, TNBS, | H&E | Gene | Large animal |
| | from mouse | from mouse | from mouse | etc. | | expression, | model of |
| | model. | model | model | | | humoral and | infectious |
| | | | | | | cellular | colitis. |
| | | | | | | responses | |

It is expected that HBP will act as an anti-inflammatory agent in both of these animal models.

### XIII. Studies Showing the HBP can Act as an Adjuvant for Anthrax Vaccines (Th1 Model)

Anthrax is a potentially lethal disease of humans and mammals (primarily herbivores) that is caused by the spore-forming bacterium, B. anthracis. The principal virulence factor of B. anthracis is a multi-component toxin secreted by the pathogen that consists of three separate gene products designated protective antigen (PA), lethal factor (LF) and edema factor (EF). Although the capsule also contributes to anthrax pathogenesis in mice, it is not clear if this is true in higher mammals such as non-human primates and rabbits. Protection against anthrax is associated with a humoral antibody immune response directed against PA and possibly EF and LF (Price, et al, 2001). The current Food and Drug Administration-approved anthrax vaccine in the United States (Product license No. 99, Biothrax or AVA) was licensed in 1972 and is produced from a non-pathogenic culture supernatant fraction that consists principally of PA adsorbed onto aluminum hydroxide (Turnbull, 2000). There are several issues regarding the reliability and quality of the Biothrax anthrax vaccine as well as the question of protective efficacy. These problems and the debate surrounding production and use of the current anthrax vaccine have prompted the interest in alternative anthrax vaccine formulations.

In this Example, we will test the immunoreactivity and adjuvant capabilities of an HBP in a vaccine model of a Th1 disease (anthrax) in A/J mice. The Sterne strain of anthracis is attenuated and can therefore be utilized in a BSL-2 model. This Example utilizes 7 groups, 5-10 mice per group, to evaluate and characterize whether injection with recombinant PA plus Heme binding protein from Ascaris can work as a vaccine for anthrax. Since in vitro tests suggest that HBP by itself can modify TH1 responses, HBP will also be tested as a vaccine by itself without rPA. The dose of HBP in these studies will be determined from previous in vitro studies. These HBP vaccines will be tested against the traditional vaccine consisting of recombinant PA (rPA) with aluminum hydroxide (Imject alum, Pierce). Mice will be inoculated with an IP injection of B anthracis Sterne spores or the use of vehicle (PBS) control. Mice will be assessed for mortality and morbidity rates as well as the development of bacteriemia over time. Control animals will receive alum alone or HBP alone without rPA.

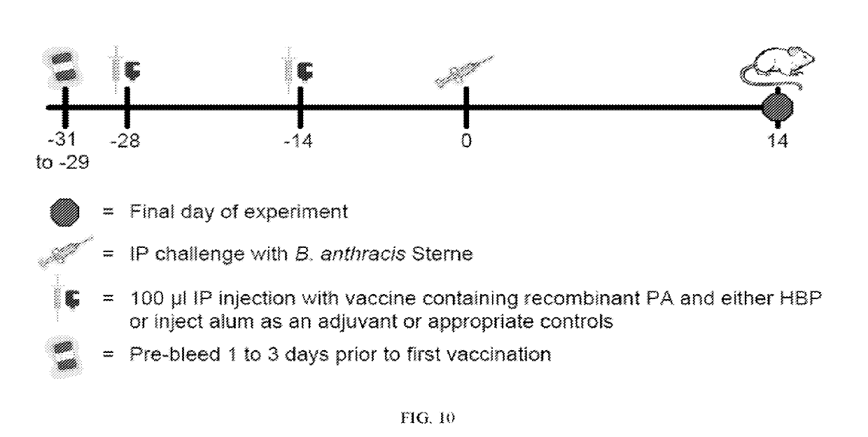

A schematic of the time course of the experiment is shown in FIG. 10.

All groups' immunological responses will be monitored by taking blood samples every 2 weeks with subsequent specific antibody measurement. The designated groups to be challenged will be injected with the pathogens on day 28 (i.e. 14 days after the final immunization). For immunological studies, 5 out of 10 mice per immunological assessment group for a total of 35 mice will be euthanized and spleen and blood samples collected at the day that their colleagues are pathogen challenged. The last 5 mice per immunological group (a total of 35 mice) will be euthanized for the same immunological analyses 14 days after IP challenge with anthrax. For a summary description of the groups see Table 2.

TABLE 2
Immunizations

| | Route of | Challenge Dose | | | | Pain | |
|---|---|---|---|---|---|---|---|
| Group | inoculation | B. anthracis | Vaccine | adjuvant | Mice | category | Rationale |
| 1 | IP | 0 | rPA | Imject alum | 5 | C | Control for alum |
| | | (PBS control) | | alone | | | immunoreactivity |
| 2 | IP | 0 | rPA | HBP alone | 5 | C | Control for HBP |
| | | (PBS control) | | | | | immunoreactivity |
| 3 | IP | 5 × 10⁵ B. anthracis | rPA | none | 10 | C | PA vaccine alone |
| | | Sterne spores | | | | | without adjuvant |
| | | (approximately 20 | | | | | |
| | | LD₅₀) | | | | | |
| 4 | IP | 5 × 10⁵ B. anthracis | rPA | Imject alum | 10 | C | PA vaccine plus |
| | | Sterne spores | | | | | Imject alum as an |
| | | | | | | | adjuvant (traditonal |
| | | | | | | | PA vaccine) |
| 5 | IP | 5 × 10⁵ B. anthracis | rPA | HBP | 10 | C | PA vaccine plus HBP |
| | | Sterne spores | | | | | as an adjuvant |
| 6 | IP | 5 × 10⁵ B. anthracis | | HBP | 10 | C | HBP alone as a |
| | | Sterne spores | | | | | potential vaccine |
| 7 | IP | 5 × 10⁵ B. anthracis | none | none | 10 | E | B. anthracis control |
| | | Sterne spores | | | | | |

| | |
|---|---|
| Total number of mice in Experiment | 60 |

⁺⁺ Vaccines will be administered on days −28 and −14 prior to infection with B. anthracis
Note:
The predicted pain category is in parenthesis next to each number. These designations are based on historic data with protein subunit vaccine.

It is expected that HBP will act as an adjuvant for this vaccine. If whole HBP can act as an adjuvant for this vaccine, we will also test Domain 1 and Domain 2 to determine if either of these sub components of HBP can provide the adjuvant affect by themselves. It is anticipated that, since Domain 2 is important for other immunological activities of this protein, it will also have an important role in the HBP adjuvant affect.

The inventors have also shown that PCF from Ascaris Helminthes, e.g. A. suum, acts as an immunotherapetuic, e.g. for treating allergic inflammation, and as a global immunosuppressant. Using the mouse model described herein, we showed that animals treated with PCF during RW sensitization have suppressed allergic responses (including suppressed allergic asthma and allergic conjunctivitis) compared to mice given RW alone. Additionally, ex vivo and in vitro analysis of PCF-treated DC demonstrated reduced activation receptor expression and cytokine production in response to either RW or LPS stimulation. These findings demonstrate that PCF alters the molecular mechanism of allergic inflammation, and further suggest that PCF can be a more general immunosuppressant that can be useful for treating, e.g., autoimmune diseases.

Some of this information was published in McConchie et al. (December, 2006), Infection and Immunity 74, 6632-6641, which is incorporated by reference in its entirety herein.

In addition, further experiments have extended these studies. Using a mouse model infected with a pathogenic species of enteropathic bacteria, Citrobacter rodentium, we showed that PCF strongly suppresses LPS-induced IL-12 production by BMDC (see FIG. 7). Because IL-12 is believed to affect Th1, this suggests that PCF can elicit a potent Th1 response. Thus, it is expected that the administration of PCF, e.g. in combination with IFN-γ, can be used to prevent a variety of Th1-mediated conditions. For example, it may be useful for the treatment of autoimmune diseases, such as inflammatory bowel diseases (IBD), multiple sclerosis (MS); and lung pathologies, such as COPD (chronic obstructive pulmonary disease, or emphysema.

Furthermore, we showed that PCF suppresses LPS induced cytokine production in LPS costimulated DCs (see FIG. 8). We injected mice intraperitoneally, with or without PCF, and examined the activation of DC six days later. PCF is thus shown to inhibit factors involved in the IgE response. These findings suggest that treatment with PCF can globally ameliorate a variety of IgE-mediated conditions in addition to allergic inflammation.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications cited above, including U.S. provisional application 60/924,537, filed May 18, 2007, and in the figures are hereby incorporated in their entirety by reference.

1. A method for suppressing inflammation in a subject in need thereof, comprising administering to the subject an effective amount of an Ascaris-derived heme-binding protein (HBP) comprising SEQ ID NO:4, or an active fragment or variant thereof.

2. The method of claim 1, which is a method for suppressing a Th-2-associated disease or condition.

3. The method of claim 2, wherein the Th-2-associated disease or condition is a form of allergic inflammation.

4. The method of claim 3, wherein the allergic inflammation is allergic conjunctivitis, allergic dermatitis, allergic eczema, allergic rhinitis, a food allergy, an eosinophil-associated gastrointestinal disorder, hyper eosinophilic syndrome (HES), eczema, or chronic urticaria.

5. The method of claim 2, wherein the subject has allergic asthma caused by ragweed.

6. The method of claim 2, wherein the subject has severe allergic asthma.

7. The method of claim 1, which is a method for suppressing a Th-1-associated disease or condition.

8. The method of claim 7, wherein the Th-1-associated disease is an autoimmune disorder.

9. The method of claim 8, wherein the autoimmune disorder is inflammatory bowel disease (IBD) or multiple sclerosis (MS).

10. The method of claim 1, wherein the HBP acts as an antioxidant.

11. The method of claim 10, wherein the subject has an intestinal inflammatory disease.

12. The method of claim 10, wherein the subject is suffering from an age-related disorder.

13. The method of claim 1, which is a method for suppressing a marker of an immunosensitive property of a cell or tissue in vitro.

14-19. (canceled)

20. The method of any of claims 1-13, wherein the Ascaris-derived HBP or active fragment or variant thereof is synthetically produced or is recombinant.

21. A pharmaceutical composition comprising an anti-inflammatory effective amount of an Ascaris-derived HBP comprising SEQ ID NO:4, or an active fragment or variant thereof, and a pharmaceutically acceptable carrier.

22. The pharmaceutical composition of claim 21, wherein the amount of the Ascaris-derived HBP or active fragment or variant thereof is effective to suppress allergic inflammation.

23. The pharmaceutical composition of claim 21, wherein the Ascaris-derived HBP or active fragment or variant thereof is synthetically produced or is recombinant.

24-25. (canceled)

26. The pharmaceutical composition of any of claims 21-23, which comprises a polypeptide that consists of SEQ ID NO:4.

27-28. (canceled)

29. A kit for treating inflammation, comprising an anti-inflammatory-effective amount of an Ascaris-derived HBP comprising SEQ ID NO:4, or an active fragment or variant thereof, or of a pharmaceutical composition of any of claims 21, 22, or 23, optionally in a vessel.

30-34. (canceled)

35. The kit of claim 29, wherein the Ascaris-derived HBP polypeptide or active fragment or variant thereof is synthetically produced or is recombinant.

36. An adjuvant composition comprising an adjuvant-effective amount of Ascaris-derived HBP or an active fragment of variant thereof.

37. The adjuvant composition of claim 36, which comprises an adjuvant-effective amount of Ascaris-derived HBP.

38. The adjuvant composition of claim 36 or 37, wherein the Ascaris-derived HBP or active fragment or variant thereof is purified from an Ascaris.

39. The adjuvant composition of claim 36 or 37, wherein the Ascaris-derived HBP or active fragment or variant thereof is produced by a synthetic or recombinant molecular procedure.

40. An immunogenic composition comprising the adjuvant composition of claim 36 and an antigen.

41. The immunogenic composition of claim 40, wherein the antigen is selected from peptides, proteins, toxoids, glycoproteins, glycolipids, lipids, carbohydrates and/or polysaccharides.

42. The immunogenic composition of claim 40, wherein the antigen is derived from a biologic or infectious organism of the animal or plant kingdom.

43. The immunogenic composition of claim 40, wherein the antigen is a whole or disrupted microorganism selected from an attenuated or inactivated virus, bacterium or parasite.

44. The immunogenic composition of claim 40, wherein the antigen is produced by synthetic or recombinant molecular procedures.

45. A method for inducing an immune response comprising administering to a subject in need thereof an effective amount of an immunogenic composition of any of claims 40-44.

46. A method for suppressing inflammation in a subject in need thereof, comprising administering to the subject an Ascaris-derived heme-binding protein (HBP) or an active fragment or variant thereof in an amount sufficient to activate bone marrow derived dendritic cells and alter cytokine production.
